(19) United States
(12) Patent Application Publication
(10) Pub. No.: US 2002/0085724 A1
(43) Pub. Date: Jul. 4, 2002

(54) STETHOSCOPE COMMUNICATION AND REMOTE DIAGNOSIS SYSTEM

(76) Inventors: JAMES A. GRASFIELD, SHARON, MA (US); DAVID E. WINSTON, ARLINGTON, MA (US)

Correspondence Address: JAMES H MORRIS, C/O WOLF GREENFIELD & SACKS PC, FEDERAL RESERVE PLAZA, 600 ATLANTIC AVENUE, BOSTON, MA, 022102211

(21) Appl. No.: 09/433,735

(22) Filed: Nov. 3, 1999

Related U.S. Application Data
(62) Division of application No. 09/019,670, filed on Feb. 6, 1998.
Application No. 09/019,670 is a continuation-in-part of application No. 08/795,755, filed on Feb. 6, 1997, now abandoned.

(51) Int. Cl. A61B 7/04

(52) U.S. Cl. 381/67

(57) ABSTRACT

An apparatus and method for transmitting a signal having a first bandwidth through a telephone line having a second bandwidth, wherein the first bandwidth includes frequencies outside the second bandwidth is disclosed. The invention also provides a method and apparatus for demodulating the transmitted signal. The system also includes a mobile unit coupled to an electronic stethoscope that communicates with a base unit using a wireless communication link. In combination with the method and apparatus for transmitting signal through a conventional telephone line, the invention allows data representative of heart and lung sounds from an electronic stethoscope that is outside the passband of a conventional telephone line to be transmitted through the telephone system for remote diagnosis and analysis.

### CROSS REFERENCE TO RELATED APPLICATIONS

This application is a division of application Ser. No. 09/019,670, filed Feb. 6, 1998, which in turn is a continuation-in-part of application Ser. No. 08/795,755, filed Feb. 6, 1997, entitled STETHOSCOPE COMMUNICATION AND REMOTE DIAGNOSIS SYSTEM, which applications are incorporated herein by reference.

### BACKGROUND OF THE INVENTION

1. Field of the Invention

The present invention relates generally to methods and apparatus for voice and data communication. In one aspect, the present invention particularly relates to methods and apparatus for voice and data communication over conventional telephone lines. In another aspect, the present invention relates to methods and apparatus for communication of electronic signals representative of biological activity.

The present invention is designed to work with and complement the electronic stethoscope disclosed in application Ser. Nos. 08/505,601 and 08/685,451, both entitled Electronic Stethoscope, the disclosures of which are hereby incorporated by reference.

2. Discussion of the Related Art

The acoustic stethoscope is typically the primary diagnostic instrument used by medical personnel, such as doctors, nurses, and emergency medical technicians to make preliminary diagnoses of heart and lung ailments and/or abnormalities. The term “user” as used in this disclosure is meant to refer to all such medical personnel. The acoustic stethoscope, however, lacks the ability to record or transmit acoustical data derived from the use of the instrument for simultaneous or subsequent analysis and diagnosis. Therefore, the user must rely solely on his or her own ears and experience to diagnose a patient when using an acoustic stethoscope.

If a physician detects the presence of a heart murmur or a breathing abnormality, or even suspects that there may be a cardiac or respiratory abnormality, then the patient is usually sent to an appropriate specialist for a more comprehensive examination. A second or confirming diagnosis is most frequently done by a second doctor (typically a cardiologist or pulmonologist depending on the diagnosis) at a location different from the location of the doctor or other medical practitioner who made the preliminary diagnosis. The second examination can take days or even weeks to be accomplished depending upon the availability of the specialist. More often than not, the specialist may be able to examine the patient either with an acoustic stethoscope alone or with an acoustic stethoscope in combination with more sophisticated test equipment to determine that the patient has no abnormality. In cases where an abnormality is detected, the specialist can make the diagnosis and supply and/or direct the appropriate treatment for the patient.

This need to send the patient to a specialist for further diagnosis presents problems. For example, transportation of a patient from an accident scene to a specialist may be impractical. In another example, the patient may be located in one country and the appropriate specialist located in another. For a variety of reasons, it may be almost impossible to have the patient travel to see the specialist or to have the specialist travel to see the patient.

Therefore, an object of the present invention is to provide a method and apparatus for voice and data communication that overcomes at least the above discussed difficulties.

Another object of the present invention is to provide a stethoscope communication and remote diagnosis system that allows communication of electronic signals representative of biological activity over conventional telephone lines.

### SUMMARY OF THE INVENTION

These and other objects that are achieved by the present invention which, in one embodiment, provides an apparatus for transmitting a signal having a first bandwidth through a telephone line having a second bandwidth, wherein the first bandwidth includes frequencies outside the second bandwidth, the apparatus comprising:

a generator circuit that generates a carrier signal having a frequency within the second bandwidth;

a modulator, coupled to the generator circuit, that modulates that carrier signal with the signal having the first bandwidth to provide a modulated carrier signal; and

an interface circuit that injects the modulated carrier signal into the telephone line.

In another embodiment of the invention, the invention provides an apparatus for demodulating a signal having a first bandwidth that has been transmitted through a telephone line having a second bandwidth, wherein the first bandwidth includes frequencies outside the second bandwidth, the apparatus comprising:

a demodulator that demodulates a received modulated carrier signal using a carrier signal to provide the signal having the first bandwidth.

In another embodiment of the invention, the invention provides a method for transmitting a signal having a first bandwidth through a telephone line having a second bandwidth, wherein the first bandwidth includes frequencies outside the second bandwidth, the method comprising the steps of:

selecting a carrier signal having a frequency within the second bandwidth;

modulating the carrier signal with the signal having the first bandwidth to provide a modulated carrier signal; and

injecting the modulated carrier signal into the telephone line.

In another embodiment of the invention, the invention provides a method for demodulating a signal having a first bandwidth that has been transmitted through a telephone line having a second bandwidth, wherein the first bandwidth includes frequencies outside the second bandwidth, the method comprising the step of:

demodulating a received modulated carrier signal using a carrier signal to provide the signal having the first bandwidth.

In another embodiment of the invention, the invention provides a communication and remote diagnosis system, comprising:

a first electronic stethoscope that provides a data signal having a first bandwidth;

a first base unit that transmits the data signal through a telephone line having a second bandwidth;

a communication link that couples the data signal provided by the electronic stethoscope to the first base unit;

wherein the first base unit includes a generator circuit that generates a carrier signal having a frequency within the second bandwidth and a modulator, coupled to the generator circuit, that modulates the carrier signal with the signal having the first bandwidth to provide a modulated carrier signal;

a first interface circuit, coupled to the first base unit, that injects the modulated carrier signal into the telephone line;

a second interface circuit, coupled to the telephone line, that receives the modulated carrier signals and provides a received modulated carrier signal; and

a second base unit, coupled to the second interface circuit, including a demodulator that demodulates the received modulated carrier signal to provide the signal having the first bandwidth.

In another embodiment of the invention, the invention provides a system for transmitting a signal having a first bandwidth through a telephone line having a second bandwidth, wherein the first bandwidth includes frequencies outside the second bandwidth, the system comprising:

a first electronic stethoscope that provides a signal having the first bandwidth;

an analog-to-digital converter, coupled to the first electronic stethoscope, that converts the signal to a first digital signal;

a compressor, coupled to the analog-to-digital converter, that compresses the first digital signal by a ratio so as to allow transmission of the first digital signal through the telephone line to provide a second digital signal; and

a first modem, coupled to the compressor, that injects the second digital signal into the telephone line.

In another embodiment of the invention, the invention provides a stethoscope to stethoscope communication system, the system comprising:

a first electronic stethoscope that provides an electronic signal representative of biological activity;

a transmitter, coupled to the first electronic stethoscope, that transmit the electronic signal;

at least one receiver that receives the transmitted electronic signal; and

at least one additional electronic stethoscope coupled to the at least one receiver.

### BRIEF DESCRIPTION OF THE DRAWINGS

In the drawings, which are incorporated herein by reference and which like elements have been given like reference characters,

FIG. 1 is an overall system block diagram of one embodiment of the invention in which multiple listeners can listen to audio signals detected by a single electronic stethoscope;

FIGS. 2A and 2B are an overall system block diagram of the present invention in which one or more remotely-located listeners can listen to biological activity detected by an electronic stethoscope;

FIG. 3 is an overall system block diagram illustrating another embodiment of the system of FIGS. 2A and 2B;

FIGS. 4A and 4B are drawings of an exemplary embodiment of the base unit illustrated in FIGS. 1-3;

FIG. 5A and 5B are drawings of an exemplary embodiment of the mobile unit illustrated in FIGS. 1-3;

FIG. 6 is a schematic block diagram of the mobile unit illustrated in FIGS. 1-3, 4A and 4B;

FIG. 7 is a schematic block diagram of the base unit illustrated in FIGS. 1-3, 5A and 5B;

FIG. 8A is a schematic block diagram of the modulator illustrated in FIG. 7;

FIG. 8B is a schematic block diagram of the demodulator illustrated in FIG. 7;

FIG. 9 is an exemplary circuit implementation of the mobile unit illustrated in FIG. 6; and

FIGS. 10, 11, 12, 13, 14, 15, and 16 illustrate an exemplary circuit implementation of the base unit illustrated in FIGS. 7 and 8; and

FIG. 17 illustrates an exemplary digital implementation of the invention.

### DETAILED DESCRIPTION

For purposes of illustration only, and not to limit generality, the present invention will now be explained with reference to its use in conjunction with the electronic stethoscope described in the aforementioned and incorporated patent applications. However, the present invention is not so limited. For example, the present invention can be used to transmit any type of data through conventional telephone lines in accordance with the frequency ranges and principles discussed herein.

With the present invention, in combination with the electronic stethoscope disclosed in the incorporated-by-reference patent applications, a doctor or medical practitioner can use the output of the electronic stethoscope to send acoustic data to other physicians or to specialists locally or remotely so that the other doctor or doctors can listen to the patient's heart and/or lungs simultaneously and make a real-time diagnosis of the patient as if the remote doctor were physically present in the same room with the patient and conducting the examination himself or herself. The advantages of this remote communication capability are numerous.

For example, one use of the present invention would be to provide doctors or other medical personnel a method by which real-time remote diagnosis of a patient's heart and/or lungs can be accomplished. The patient can be in the presence of any basically trained medical practitioner. Using the electronic stethoscope in combination with the present invention, the medical practitioner can send the acoustic data over any single conventional telephone line anywhere in the world to a second doctor or medical practitioner using another electronic stethoscope for remote real-time examination of the patient by the remotely located doctor or medical practitioner.

Another use of the present invention is to allow a remotely-located doctor to monitor and/or examine the heart and/or lungs of a patient in real-time when the patient is anywhere outside of the physician's physical presence (e.g. at home, in a nursing or convalescence home, in the field, etc.) using the electronic stethoscope in combination with the present invention and a single conventional telephone line.

The present invention allows real-time bidirectional communication of both voice and audio data derived from the electronic stethoscope between a local site and a remote site using a single conventional telephone line. So called “plain old telephone service” (POTS) typically has a bandwidth of 300 Hz to 3400 Hz which is nominally designed to carry voice transmissions. The frequency range of heart and lung sounds, however, is typically in the range of 20 Hz to 1600 Hz, and a significant portion of the audio energy of the heart sounds and some lung sounds fall substantially below the 300 Hz lower limit of POTS.

Since POTS is the lowest common denominator among various types of telephone service, the present invention advantageously allows audio signals outside the range of the normal POTS frequency range to be clearly transmitted. This avoids the need for more expensive broader bandwidth communication lines that are not universally available. The present invention thus allows transmission of heart and lung sounds over even the most basic telephone service to allow precise and faithful (and unadulterated or undistorted) heart and/or lung sounds to be transmitted from one electronic stethoscope to another for accurate diagnosis in real time. In addition, the medical practitioners can communicate with each other during the examination in real time to facilitate the direction of the placement of the chestpiece on the patient and other aspects of the examination by the doctor at the remote site.

One aspect of the present invention includes a method for modulating the audio data derived from the stethoscope into the POTS passband for transmission over the telephone line. At the receiving end of the transmission (i.e., the remote site) the modulated audio signal is demodulated to recover the original stethoscope audio signal. Voice signals are transmitted unmodulated onto the telephone line to preserve the maximum bandwidth for these signals. Discrimination circuitry and logic in the base units at the local site and the remote site determine if the data transmitted over the telephone line is voice or stethoscope data.

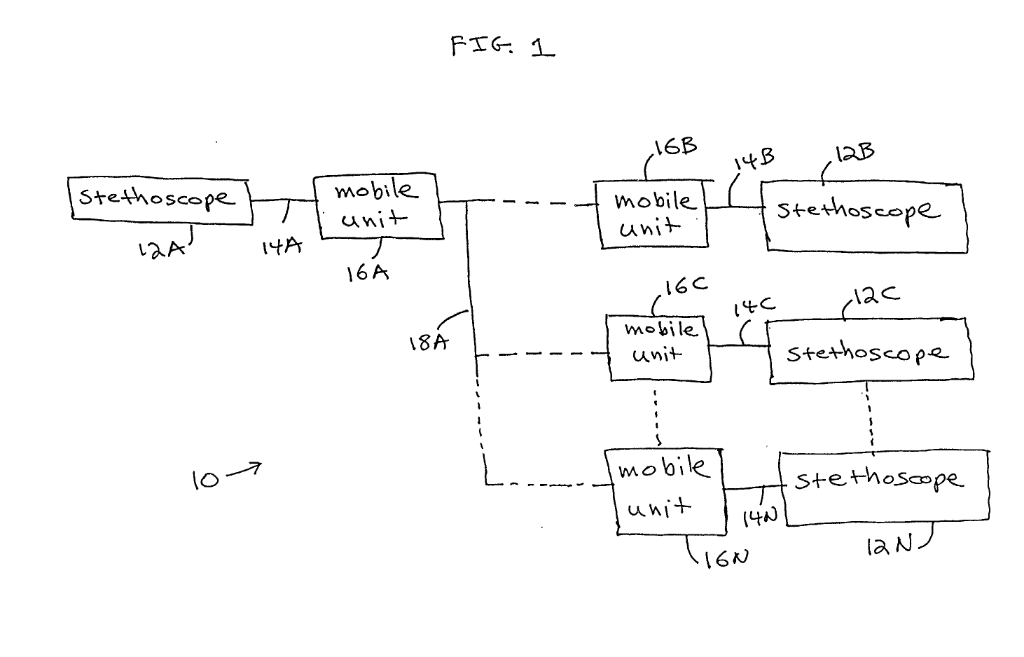

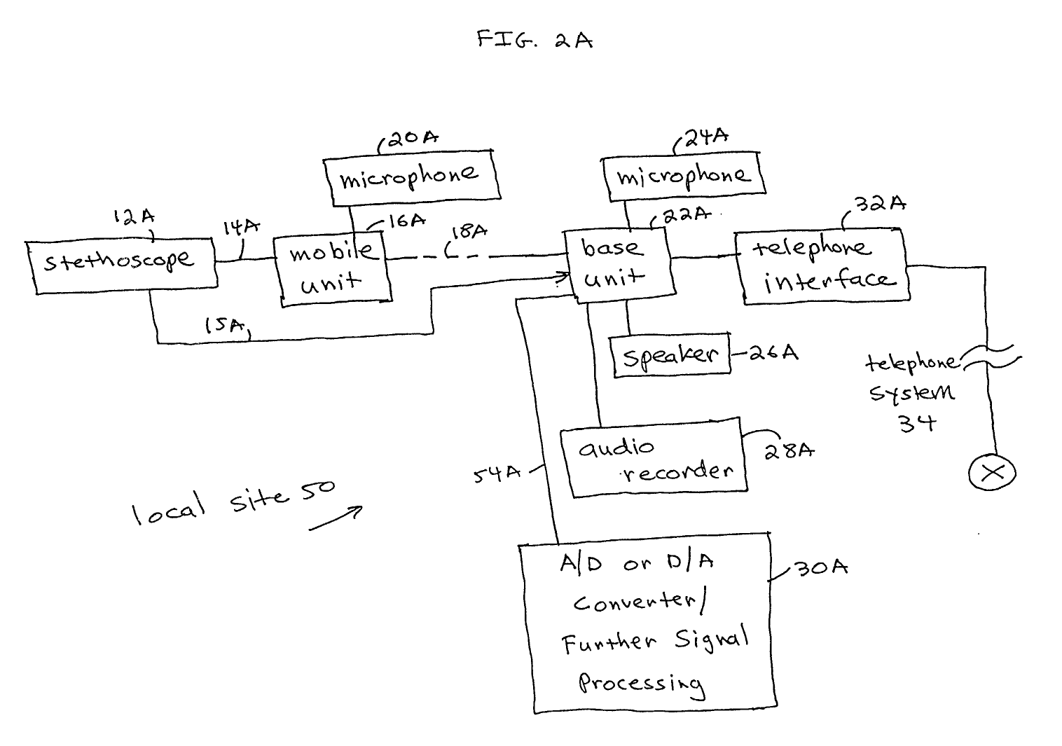

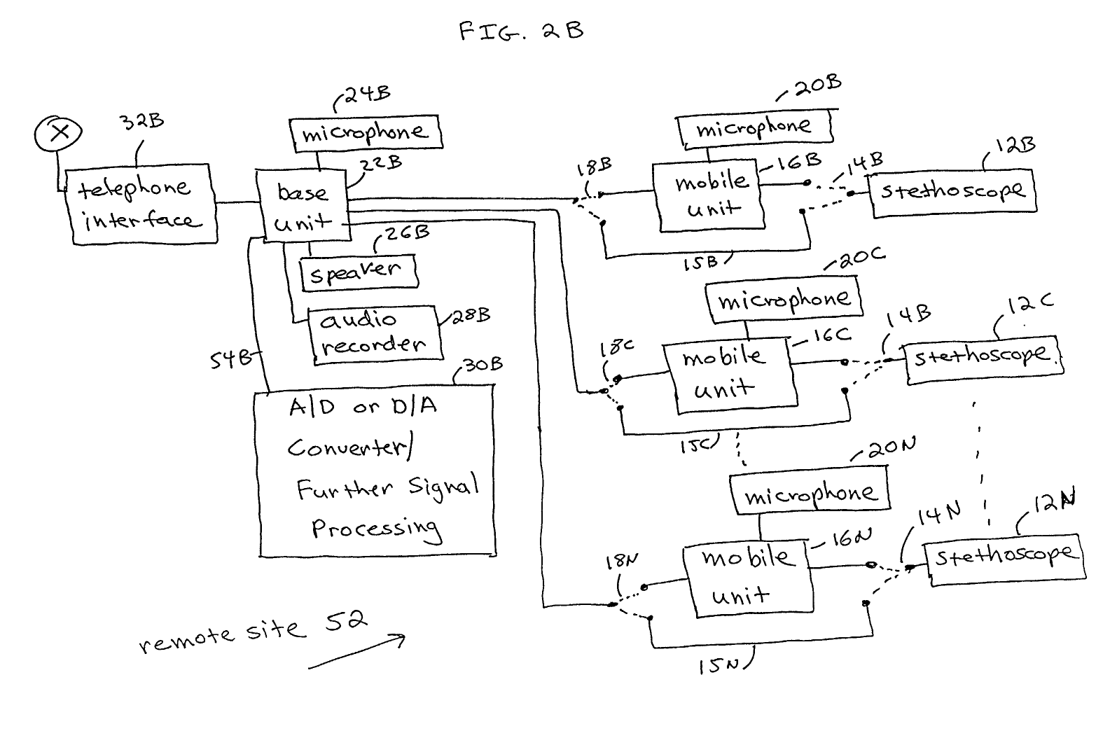

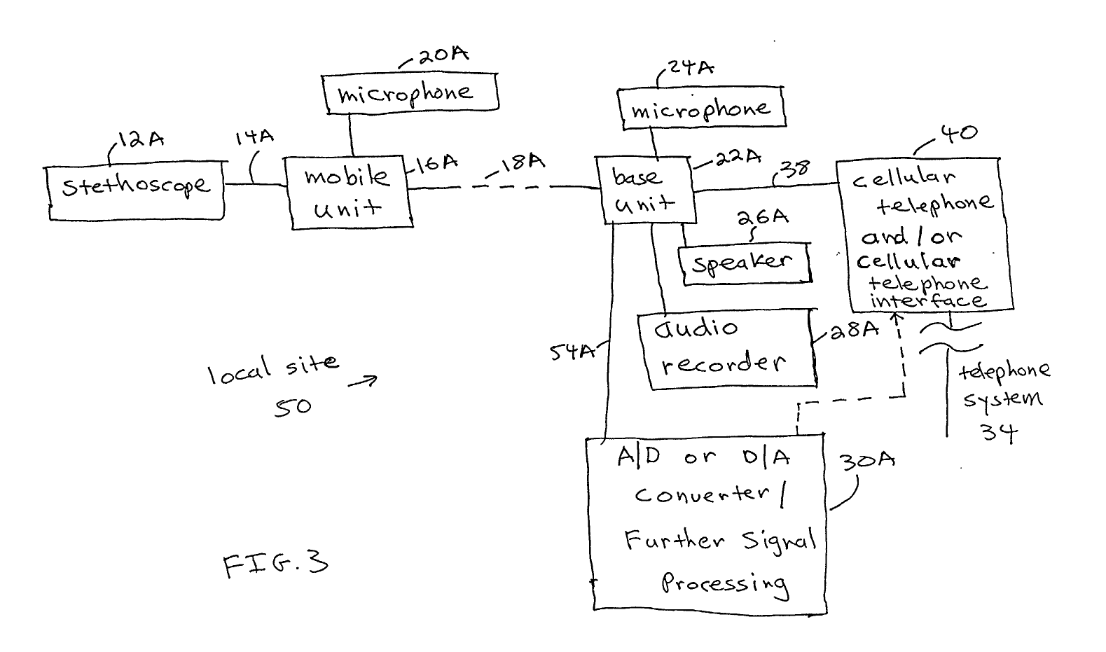

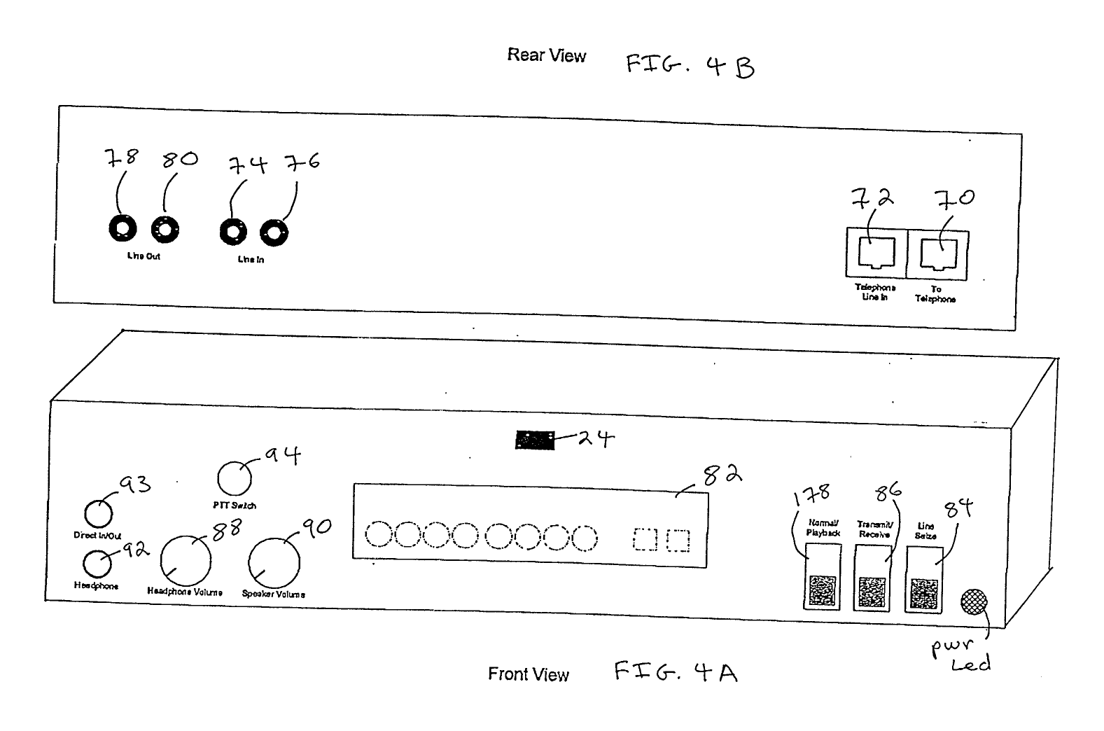

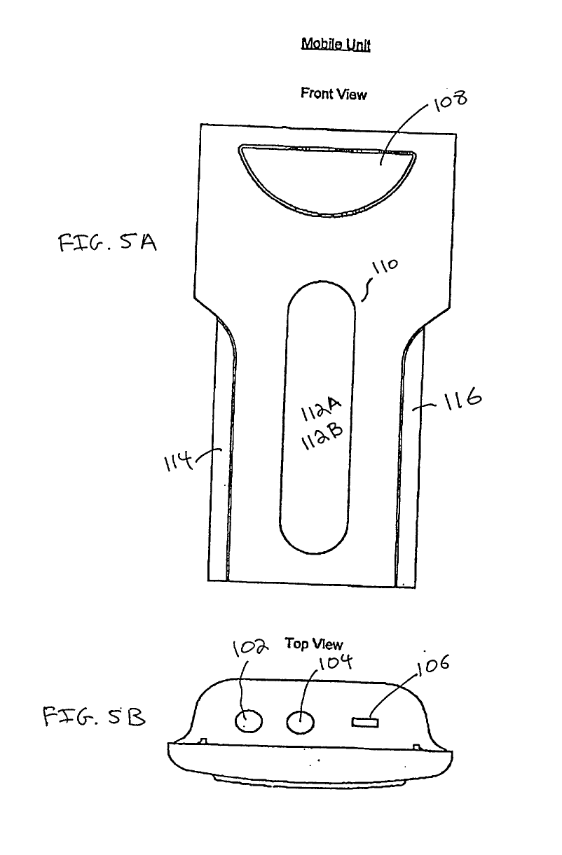

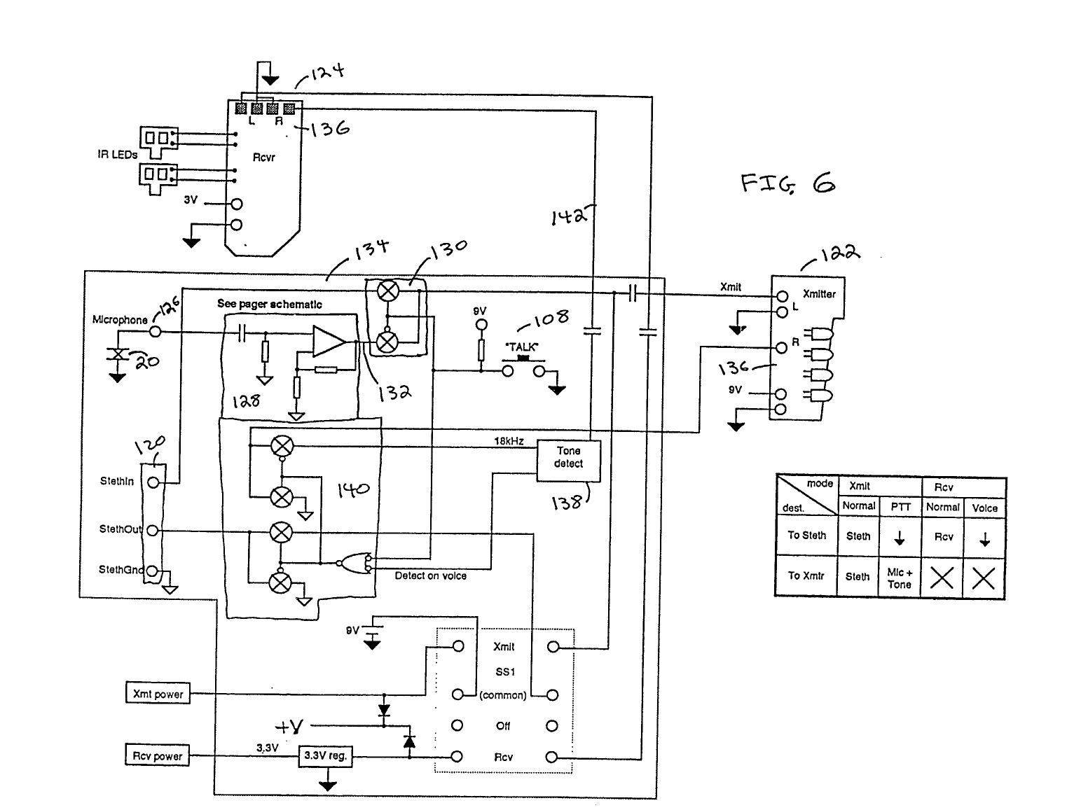

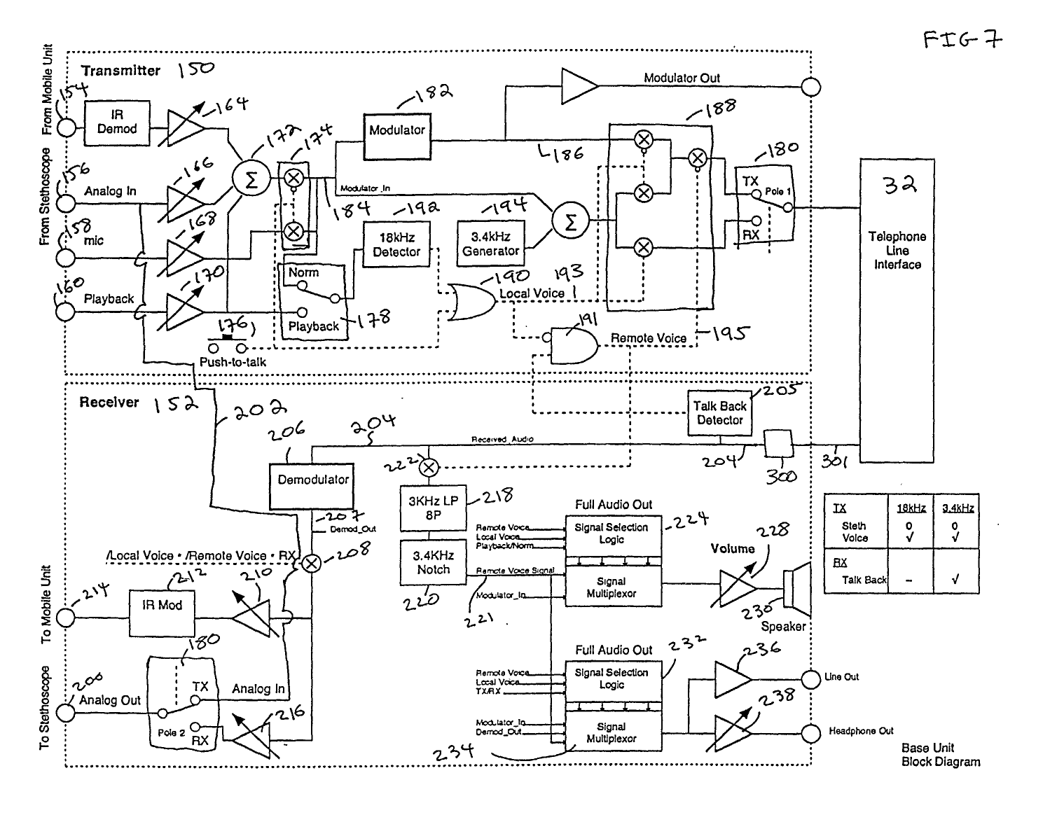

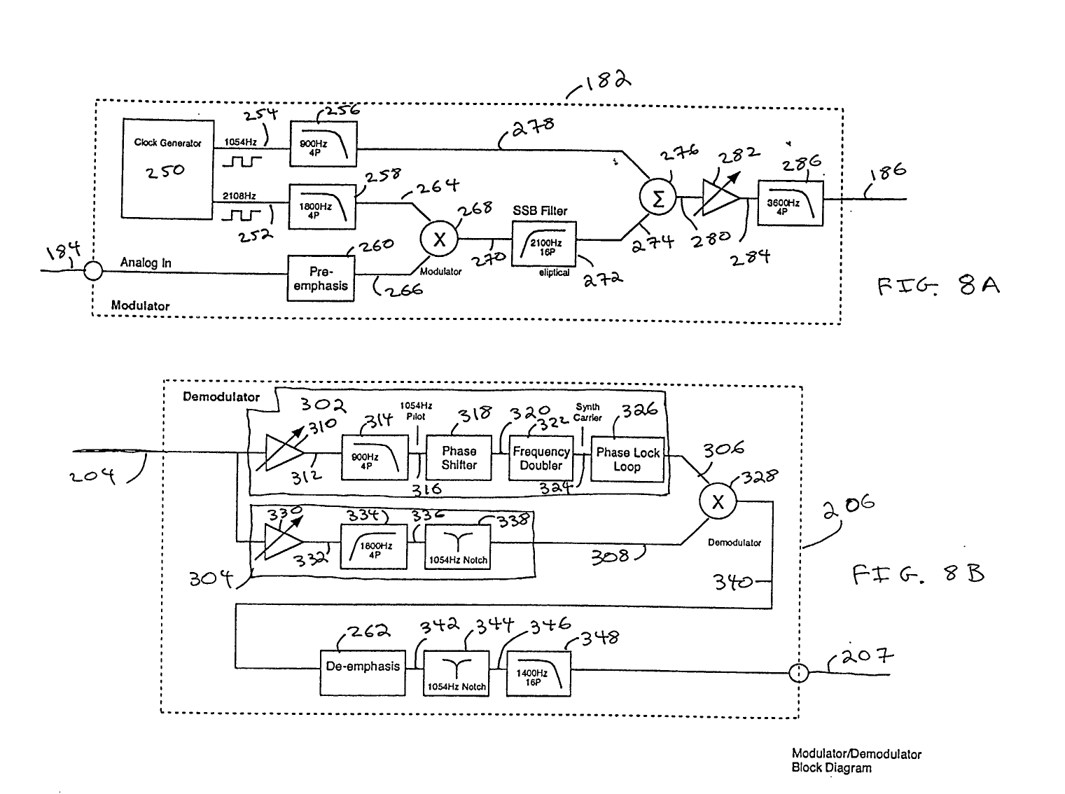

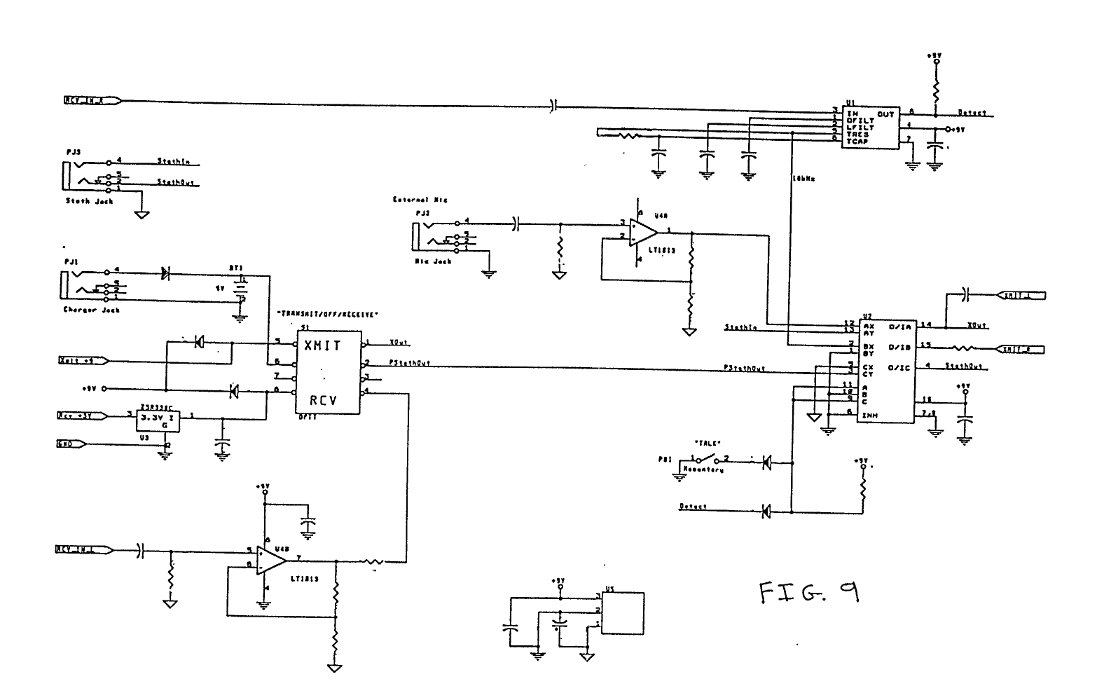

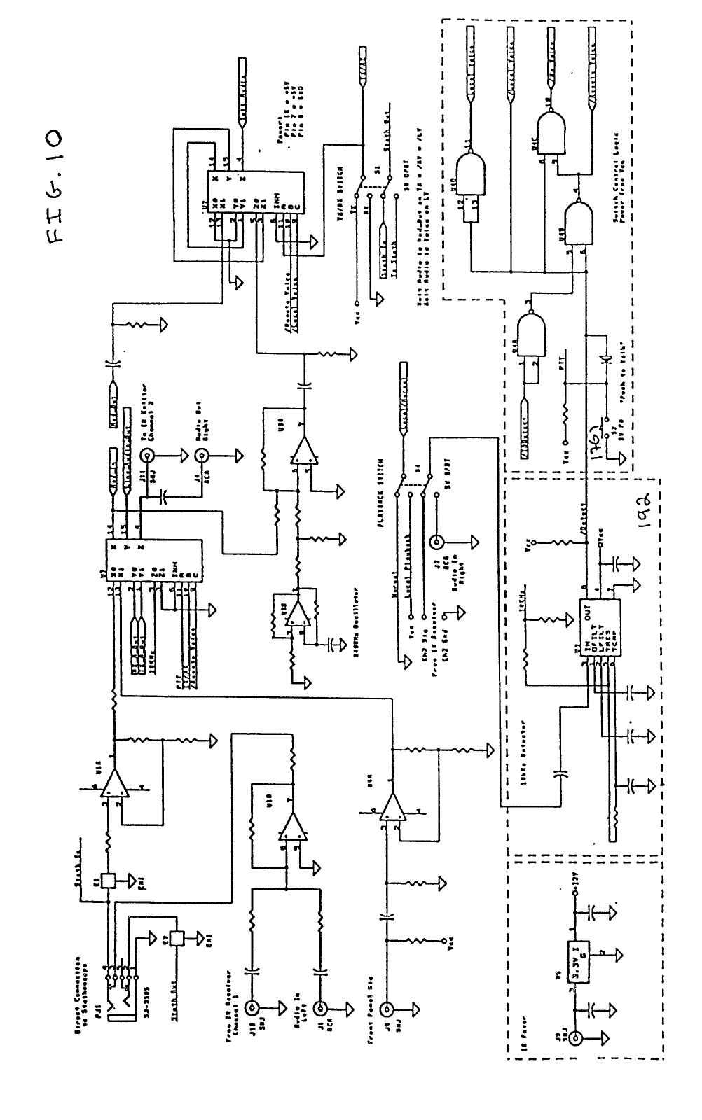

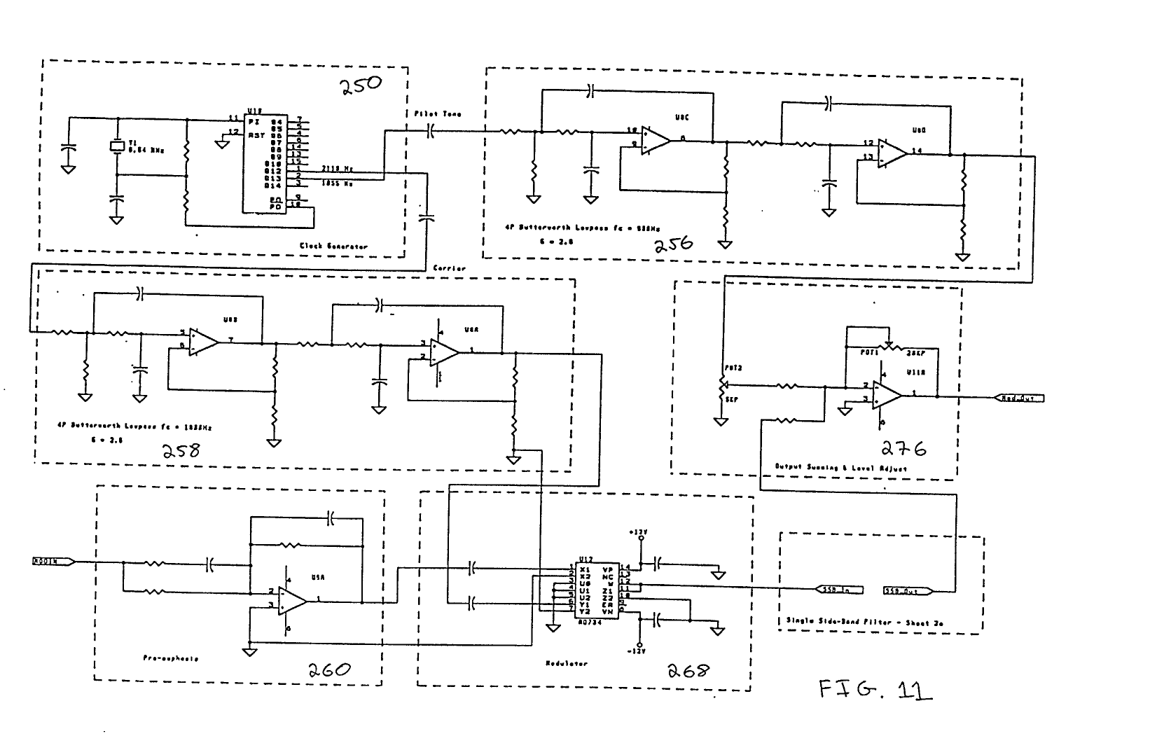

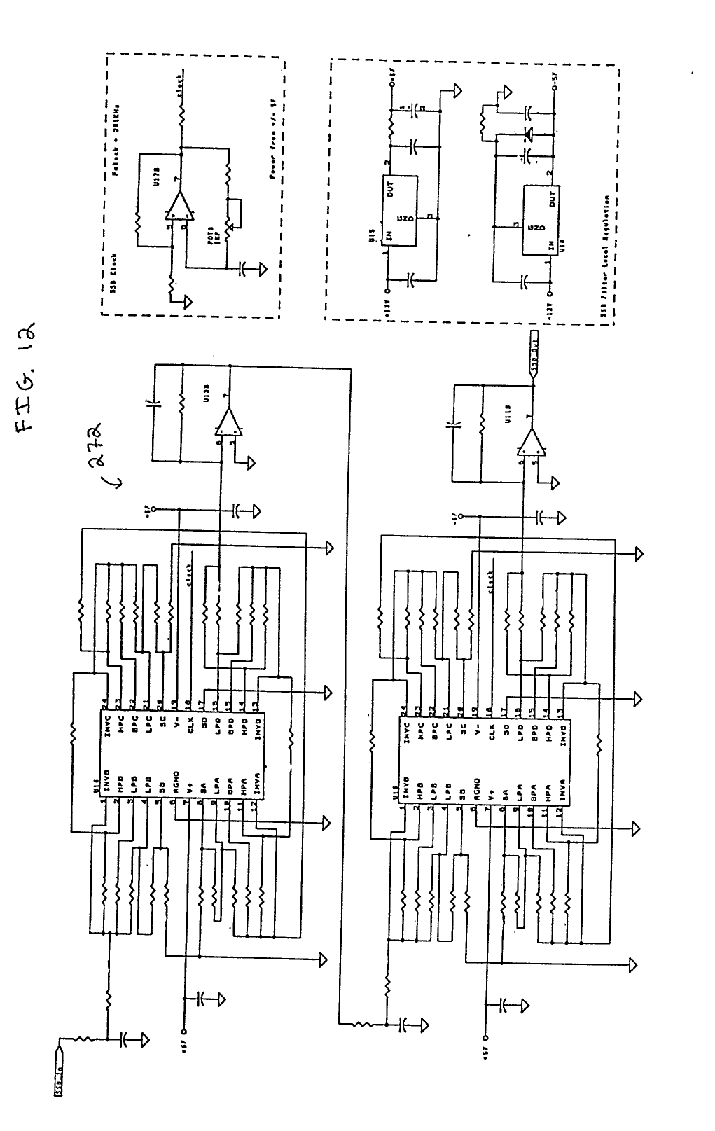

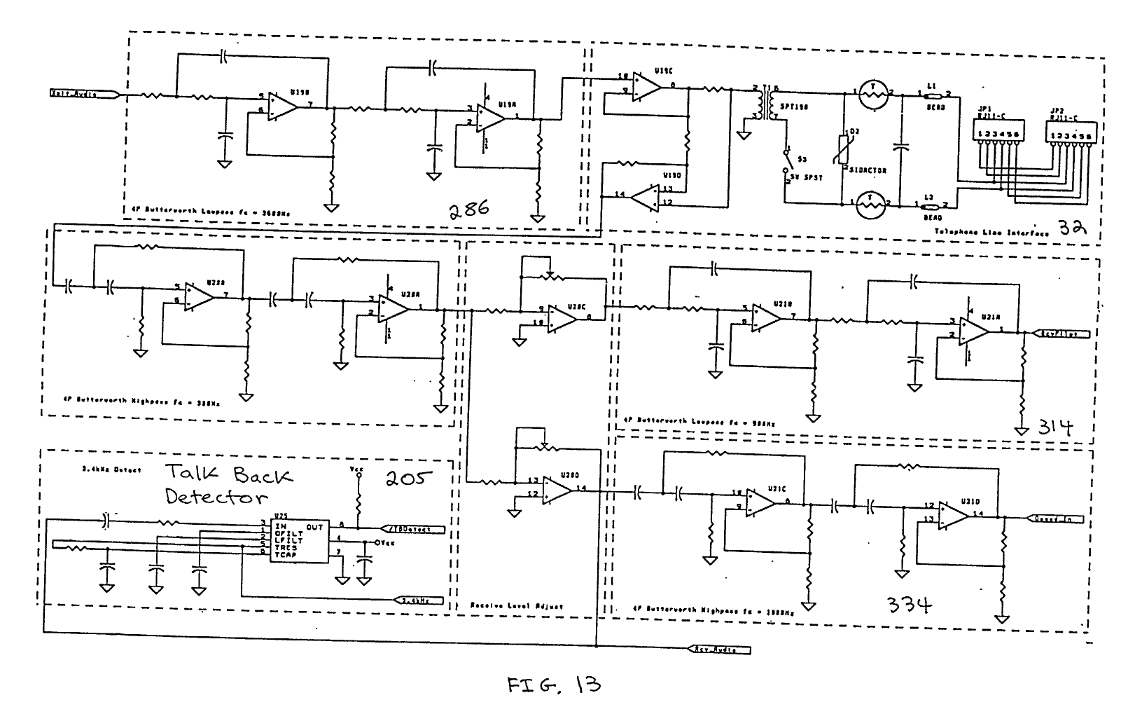

Reference is now made to FIG. 1, which figure illustrates a first embodiment of the present invention. In FIG. 1, the electronic stethoscope 12A is coupled via communication link 14A to a mobile unit 16A. Communication link 14A is connected to, for example, transceiver interface 114 illustrated in FIG. 12 of the incorporated by reference applications. Communications link 14A transmits the electronic data from stethoscope 12A to mobile unit 16A. Mobile unit 16A includes a wireless transmitter capable of transmitting the signals received from the electronic stethoscope over wireless communications link 18A. Wireless communications link 18A may be a radio frequency (RF) communications link or an infrared communication link (IR). As noted in the incorporated-by-reference applications, the IR link is preferred because of reduced potential for interference.

Mobile unit 16A includes both transmitting and receiving circuitry. Although illustrated in FIG. 1 as a separate device, the circuitry of mobile unit 16A could be incorporated directly into the electronic stethoscope 12A. Alternatively, mobile unit 16A can be a small device suitable for being clipped to a user's belt and communication link 14A can simply be a cable that plugs into transceiver interface 114 in stethoscope 12A.

A number of mobile units 16B-16N are respectively connected via communications links 14B-14N to stethoscopes 12B-12N. Mobile units 16B-16N are configured as receivers that receive the electronic signals transmitted by mobile unit 16A over wireless communications link 18A.

When configured in this manner, the present invention may be used to allow multiple listeners to hear, simultaneously and exactly, the acoustic signals being detected by electronic stethoscope 12A. This configuration allows a number of users to simultaneously participate in diagnosis or alternatively allows a single teacher to instruct a number of students in auscultation.

This embodiment of the present invention provides a precision methodology for teaching auscultation to students. The current methodology for teaching auscultation on live patients involves a teaching physician using an acoustic stethoscope to hear an abnormal heart or lung sound. The teaching physician applies the chestpiece of his or her stethoscope to the patient in a certain position and with a specific degree of pressure to best hear the desired sound. Each student, in turn, then listens with his/her respective acoustic stethoscopes to try to replicate the procedure that enabled the teaching physician to hear the desired sound. The teaching physician, however, cannot directly monitor what each student is hearing, for the stethoscope only has a single set of binaurals which are placed in the student's ears. Because there are many subtle variables which can enable a listener to hear a specific sound, there is no guarantee that the student will hear the same sound (or any sound at all) as the teaching physician. Using the present invention in one mode of operation, the teaching physician can place the chestpiece of his/her electronic stethoscope on the patient and locate the optimal location and position for best auscultating the desired sound. Each student can then listen through his/her own electronic stethoscope and hear exactly the sound that the teaching physician is hearing in real time through the local infrared transmission path of the present invention. There is no limit to the number of students who can listen simultaneously. In this mode of operation, each student can independently adjust the volume of the signal being received by his/her electronic stethoscope and can independently adjust the mode (i.e. which controls the selectable frequency ranges and/or other processing circuitry incorporated in the electronic stethoscope) in which the sound is best heard.

Reference is now made to FIGS. 2A and 2B which figures illustrate an overall system block diagram of the present invention in which one or more remotely-located listeners can listen to biological activity detected by an electronic stethoscope.

The system in FIGS. 2A and 2B includes two basic sections: a “local site” 50 and a “remote site” 52. The local site and remote site are connected by a conventional telephone line over the conventional telephone system 34. In a typical use, a patient would be located at the local site and one or more doctors, specialists, and diagnosticians would be located at the remote site 52.

In the system illustrated in FIGS. 2A and 2B, a base unit 22A and a base unite 22B are respectively coupled to the local and remote ends of telephone system 34 using telephone interfaces 32A and 32B. The addition of base units 22A and 22B thus allows the system of FIG. 1 to be expanded so that the doctor or medical practitioner actually examining a patient with stethoscope 12A can transmit these signals detected along with his or her comments over a conventional telephone line in telephone system 34 to one or more other medical practitioners, doctors, or students at the remote site 52.

The system of FIGS. 2A and 2B has several additional features. At either the local site 50 or the remote site 52, a mobile unit 16A-16N in combination with a wireless communications link 18A-18N can be used to transmit signals between the base unit and the electronic stethoscope. Alternatively, a direct-wired communications link 15A at the local site or 15B-15N at the remote site can be used to connect a respective stethoscope and base unit. One skilled in the art will appreciate that although separate communication links 18B-18N have been illustrated in FIGS. 2A and 2B, if base unit 22B is being used in the wireless communications mode, the base unit simply broadcasts a signal that is then received by each of the mobile units 16B-16N.

An important feature of the system illustrated in FIGS. 2A and 2B, which will be explained in greater detail hereinafter, concerns the use of conventional telephone lines. As a result of the processing circuitry used in the mobile units 16 and the base units 22, the present invention is able to use a conventional telephone line having a frequency response in the range of 300 Hz-3400 Hz to transmit both voice and stethoscope data with a degree of fidelity that allows a medical practitioner or doctor at the remote site to clearly hear the acoustic signals being detected by the stethoscope at the local site to accurately diagnose a patient.

Another feature of the system of FIGS. 2A and 2B is the inclusion of microphones 20A-20N, 24A, and 24B. These microphones allow the users at the local site and the users at the remote site to communicate and comment upon the stethoscope data being transmitted. A speaker 26A and a speaker 26B allows the users at the local site and the remote site to hear voice communications. An audio recorder 28A at the local site 50 and an audio recorder 28B at the remote site are respectively coupled to the base unit 22A and the base unit 22B. Either the remote site or the local site can record the stethoscope data that is being transmitted or received for purposes of later diagnosis and analysis, as well as for comparison to earlier or subsequent auscultations in order to determine a patient's progress.

Base unit 22A is also capable of sending an analog stethoscope signal to processing block 30A for further signal processing and for receiving a signal from processing block 30A. Processing block 30A may contain, for example, an analog-to-digital converter allowing the signal to be digitized and provided to a computer for, for example, digital filtering and analysis. Alternatively, processing block 30A could, via a digital-to-analog converter convert a digital signal to an analog signal and then the analog signal over communications link 54A to base unit 22A for transmission over the telephone system 34 to the remote site 52.

Reference is now made to FIG. 3, which figure illustrates an overall system block diagram illustrating another embodiment of the system of FIGS. 2A and 2B.

In FIG. 3, base unit 22A at the local site 50 is coupled, by a communications link 38 to a cellular telephone or cellular telephone interface 40. The cellular telephone is then coupled, via conventional technology into telephone system 34. The embodiment illustrated in FIG. 3 is advantageous in that the base unit does not have to be plugged directly into a conventional telephone line in telephone system 34, but can instead be directed into a cellular telephone for transmission over telephone system 34. This is particularly advantageous in, for example mobile systems and emergency response vehicles. For example, a base unit and cellular telephone could be located in an ambulance. When responding to an emergency, an emergency medical technician in the ambulance, using stethoscope 12A could examine a patient at an accident scene and using mobile unit 16A transmit the stethoscope data to base unit 22A located in the ambulance. Base unit 22A can then transmit this data to cellular telephone 40 which can in turn transmit the stethoscope data into telephone system 34 via conventional cellular telephone technology to allow the emergency medical technician, via the mobile unit to communicate with a triage doctor located at remote site 52.

When used in this configuration, wireless communications link 18A could be an RF communications link which may be more useful in situations where the patient in an emergency situation may be outside or at a relatively far distance from the ambulance containing base unit 22A.

Alternatively, the base unit and the cellular telephone could be carried by the emergency medical technician to a victim in an emergency.

Reference is now made to FIGS. 4A and 4B which illustrate, respectively, a front view, and a rear view of base unit 22. Base unit 22 is connected to a telephone line in a manner similar to a conventional facsimile or answering machine using jacks 70 and 72. Line-in jacks 74, 76 allow signals to be received by the base unit from, for example, audio recorder 28 or signal processing block 30. Line-out jacks 78, 80 allow stethoscope data and voice to be provided to audio recorder 28 or signal processing block 30.

The front panel illustrated in FIG. 4A, includes a microphone 24 and an IR or RF transmitter/receiver 82. Line seize switch 84 controls a telephone interface within base unit 22 to allow the base unit to maintain the connection between the local site and the base unit at the remote site even when the telephone connected to jacks 70 and 72 is hung up. A telephone may be used to establish the connection between the local site and the remote site. Transmit/receive switch 86 places the base unit in the transmit or receive mode depending on whether it is being used at the local site or the remote site. Headphone volume control 88 and speaker volume control 90 are provided to allow control of headphones connected to headphone jack 92 or speaker 26 contained within the base unit. A jack 93 is provided for connection to direct wired communications link 15.

A push-to-talk switch 94 is provided on the base unit to allow a user to interrupt the stethoscope data so that voice comments can be transmitted over the telephone line. A switch 178 is used to, among other things, defeat the voice override capability from the mobile unit so as to allow a previously recorded signal to be transmitted uninterrupted.

Reference is now made to FIGS. 5A and 5B, which figures respectively illustrate front and top views of one embodiment of mobile unit 16. Mobile unit 16 includes a microphone jack 102 that allows an external microphone, such as a lapel microphone, to be connected to the mobile unit. Another jack 104 is provided for connection to communications link 14 to allow the mobile unit to transmit and receive stethoscope data to and from electronic stethoscope 12. A mode switch 106 is used to place mobile unit 16 in one of its two operational modes, transmitting or receiving and to turn off power to the mobile unit.

A push-to-talk switch 108 is provided that allows the mobile unit to interrupt the stethoscope data to transmit voice comments using microphone 20.

If RF communication is being used between the mobile unit and the base unit, an RF transmitter/receiver 110 is provided. Alternatively, if IR transmission is being used, two IR receivers 112A and 112B and two IR transmitters 114 and 116 are provided in mobile unit 16. One transmission channel is used to transmit stethoscope and voice data and the second transmission channel is to transmit a marker tone. The use of two transmission channels will be explained in greater detail hereinafter.

Reference is now made to FIG. 6, which figure is a block diagram of mobile unit 22 illustrated if FIGS. 1-3. The mobile unit is a small, typically belt-worn device similar in size to a standard pager. Mobile unit 22 includes a stereo audio jack 120 for interconnection to the electronic stethoscope via cable 14. A two channel bidirectional infrared transceiver including transmitter 122 and receiver 124 provide wireless bidirectional communication with a base unit or with other mobile units and their attached stethoscopes. The bidirectional transceiver allows stethoscope data to be transmitted from or received by the mobile unit. An input jack 126 allows microphone 20 to be connected to the mobile unit. A preamplifier section 128 amplifies and buffers the signal from microphone 20.

When set in the transmission mode, stethoscope data is normally transmitted from the mobile unit. When the user wishes to talk, he or she pushes talk button 108 which causes multiplexer 130 to select the voice output on line 132 from preamplifier section 128 rather than the stethoscope data on line 134. When the momentary push-to-talk button 108 is released, switch 130 selects the stethoscope data on line 134 and deselects the output of preamplifier section 128.

A second transmission and reception channel is also provided in mobile unit 22 for detecting when voice rather than stethoscope data is being transmitted. The second transmission channel 136 is connected to an 18 KHz tone generator and detector 138. When push-to-talk button 108 is pressed, the 18 KHz tone is provided via switch 140 to transmitter 122. A base unit or another mobile unit receiving the 18 KHz tone responds to the tone by switching off the stethoscope data to allow the voice signal to be received and passed unprocessed by the circuitry used to transmit the stethoscope data.

Alternatively, if mobile unit 22 is in the receive mode, receipt of the 18 KHz tone on line 142 causes tone generator and detector 138 to switch off the stethoscope data being provided through switch 140 to jack 120 so that the user can hear the unprocessed voice signal through the speaker 26 of the base unit that the mobile unit is communicating with.

Reference is now made to FIG. 7, which figure is a schematic block diagram of the base unit illustrated in FIGS. 2 and 3. The base unit contains: a two-channel IR receiver for the reception of wireless transmission of stethoscope and voice signals from the mobile unit and electronic stethoscope; discrimination circuitry to distinguish voice signals from the mobile unit microphone; a modulation system for frequency shifting stethoscope signals into the telephone network bandwidth at the transmitting end of the communication; signal switching circuitry for directing voice signals directly to the telephone line interface or stethoscope signals through the modulator prior to connection to the telephone line interface; a demodulation system for recovery of the stethoscope signals at the receiving end of the communication; a detector for identifying return voice communication from the receiving end of the communication directed to the transmitting end; and signal switching circuitry for directing return voice signals to an internal amplifier and loudspeaker and for directing stethoscope signals to a jack and to an IR transmitter for wireless transmission to a mobile unit and electronic stethoscope.

The base unit contains a transmitter section 150 and a receiver section 152. Coupled to the transmitter section 150 and the receiver section 152 is the telephone interface 32.

Transmitter section 150 is used to transmit voice and stethoscope data over the conventional telephone line to a corresponding receiver section 152 located in a base unit at the remote site.

Receiver 152, located at a remote site, is used to receive voice and stethoscope data from another transmitter section 150. Receiver section 152 contains circuitry for transmitting stethoscope data to a stethoscope via a direct connection or via a mobile unit 16 and an IR or RF interface. Receiver section 152 also contains circuitry for playing received voice data through a local speaker.

Transmitter section 150 has, in the illustrated embodiment, four inputs. Input 154 receives the wireless transmission from a mobile unit 16. Input 156 receives the stethoscope data from communications link 15 when a direct-wired connection is used. Input 158 receives an audio input from microphone 24A which may be worn by the user if the direct wire communications link 15A is being used or may be located within the base unit itself. Input 160 receives an audio signal from, for example, audio recorder 28 or processing block 30.

The stethoscope data from input 154 is sent through an IR demodulator 162 and amplifier 164. The signals received on inputs 156, 158, and 160 are respectively buffered and amplified by amplifiers 166, 168, and 170. The outputs of amplifiers 164, 166, and 170 are coupled to a summing circuit 172. The output of amplifier 168 and summing circuit 172 are coupled to a multiplexer 174. In response to activation of momentary push-to-talk to switch 176 or activation of 18 KHz tone detector 192, multiplexer 174 selects either stethoscope data from inputs 154, 156, and 160 or voice data from input 158 for transmission over the telephone line. In normal operation, stethoscope data is always selected and activation of switch 176 serves to interrupt the stethoscope data to allow voice data transmission.

A switch 178 is provided that overrides the default normal operation. When switch 178 is placed in the playback position, the input to 18 KHz marker tone detector 192 is switched to the playback source. This activates control gates 190 and 191 that control multiplexer 188 to select only the signal on line 187 so as to allow only transmission of audio signals connected to input 160 and to disable transmission of data from inputs 154, 156, and 158.

A mode selection switch 180 is provided for selecting between a transmit and receive mode of operation for the base unit.

In the transmit mode of operation, the signal from multiplexer 174 is sent to modulator 182 on line 184. Modulator 182 frequency shifts the stethoscope signals having a frequency range of approximately 20 Hz to 1600 Hz into the passband frequency range of the POTS line of approximately 300 Hz to 3400 Hz. The output of modulator 182 is sent on line 186 through a multiplexer 188 to telephone line interface 32 for transmission over the telephone network.

In the transmit mode, if the user at the local site desires to engage in voice communication with the remote site, he or she presses push-to-talk switch 176. Push-to-talk switch 176 activates gate 190 to provide a control signal on lines 193 and 195 to multiplexer 188. This signal causes multiplexer 188 to interrupt the signal supplied by modulator 182 and to instead transmit the voice signal from input 158 and a 3.4 KHz marker tone generated by tone generator 194 directly into the telephone line interface 32. If the user is using a mobile unit 16, he or she presses push-to-talk switch 108 which causes an 18 KHz marker tone to be transmitted on one of the two IR channels. This signal is received, along with the stethoscope audio, by demodulator 162. The 18 KHz marker tone is detected by tone detector 192 and control gates 190, 191, and multiplexer 188 in the same manner as described in connection with push-to-talk button 176.

In the transmit mode, with switch 180 set to the TX position, the analog output at terminal 200 is connected via connection 202 to input terminal 156.

In the receive mode of operation, when the base unit 22 is located at the remote site 52 and is operated as a receiver, switch 180 is moved to the RX position. In this mode, the base unit defaults to reception of stethoscope data but switching is provided, as will be explained, to allow the user at the remote site or the user at the local site to interrupt the stethoscope data in order to conduct voice communications between the local site and the remote site.

In receive mode, receiver section 152 receives the signal from the local site on line 301. The modulated stethoscope audio signal on line 301 is provided to a high pass filter 300. Filter 300 is a high pass four pole Butterworth filter having a corner frequency at approximately 300 Hz. High pass filter 300 reduces the amount of power-line coupled 60 Hz noise that may be present on the incoming signal. From filter 300, the modulated stethoscope audio signal is provided on line 204. The default mode of operation of receiver section 152 assumes that the received audio signal is stethoscope data. The received audio signal is routed to the demodulator 206 which demodulates the stethoscope signal that had been modulated by modulator 182. The demodulated stethoscope data passes through switch 208, amplifier 210, IR modulator 212 and terminal 214 for transmission to a mobile unit 16. The demodulated stethoscope data is also routed through variable gain amplifier 216 and terminal 200 if direct wired communications link 15 is being used to transmit the stethoscope data to the stethoscope. Switch 208, under control of the voice switching logic, turns off the stethoscope data if push-to-talk button 176 located in the base unit at the local site or the base unit at the remote site is pushed so as to interrupt the stethoscope audio and allow voice communication.

As noted previously, a 3.4 KHz marker tone is added to a transmitted voice signal. The 3.4 KHz tone is also received on line 301. The received voice and 3.4 KHz tone is filtered by low pass filter 218 and a notch filter 220 having a 3.4 KHz notch. Low pass filter 218 and notch filter 220 reduce the amplitude of the 3.4 KHz marker tone to provide a voice signal on line 221 substantially free of the 3.4 KHz marker tone. The voice signal on line 221 is provided to signal multiplexer 226 that, under control of selection logic 224, selects the signal for routing through variable gain amplifier 228 and into speaker 230 so that these voice or playback signals can be heard by the user at the remote site. These same signals are also routed through signal multiplexer 234 under control of selection logic 232, amplifier 236, variable gain amplifier 238 to provide signals that can be listened to with headphones or recorded or routed to some other device.

If the 3.4 KHz marker tone is detected by a talk back tone detector 205, then switch 208 is turned off and switch 222 is turned on to allow data, other than stethoscope data, such as a voice signal from the local site, or a playback signal from the local site to be selected by the receiver for routing to speaker 230 or the line-out or headphone jacks.

The overall operation of the system including base units 22A and 22B is as follows. In a typical circumstance, the base unit at the local site 50 is configured as the transmitter and the base unit at the remote site 52 is configured as the receiver. In their default mode of operation, the base unit 22 transmits stethoscope data received directly from stethoscope 12A or via wireless connection 18A across the telephone system 34 to the base unit 22B located at the remote site 52. If the user at the local site 50 wishes to speak with the user at the remote site 52, he or she presses push-to-talk switch 108 located in the mobile unit or push-to-talk switch 176 located in the base unit 22A. Push-to-talk switch 176 in the base unit causes multiplexer 188 to choose the voice signal from terminal 158 for transmission over the telephone line rather than the signal from the output of modulator 182. A 3.4 KHz tone from tone generator 191 is added to the transmitted, unmodulated voice signal. This 3.4 KHz tone is detected by the corresponding receiver section 152 located in the base unit 22B at the remote site 52 and causes base unit 22B to turn off the demodulated stethoscope data being supplied by demodulator 206 to terminals 200, 214 and to instead route the received audio through amplifiers 228, 236, 238 to speaker 230 or the line-out or headphone outputs. As long as the push-to-talk button 176 is pressed, the 3.4 KHz tone will continue to be transmitted across the telephone line. As soon as push-to-talk button 176 is released, the 3.4 KHz tone is removed. Absence of the 3.4 KHz tone causes multiplexer 188 in transmitter section 150 to select the modulated stethoscope data on line 186 for transmission over the telephone line. In the same manner, absence of the 3.4 KHz marker tone causes the receiver section 152 to turn off the signal to speaker 230 and to supply the demodulated stethoscope data to terminals 200, 214.

Presence of the 3.4 KHz tone directs the system to transmit voice signals (or signals other than stethoscope data, such as recorded signals input at terminal 160) and absence of the 3.4 KHz marker tone signal controls the system to transmit stethoscope data through telephone system 34.

When the user at the remote site 52 desires to engage in voice communication with the user at the local site 50, the operation of the system is the same as described in connection with the user at the local site 50.

As an alternative to the use of push-to-talk buttons, microphones 20 and 24 could be controlled using voice activated switching.

The 3.4 KHz marker frequency tone was chosen so as to be within the passband of a POTS line, but so as to not to interfere with normal communications. Obviously, other frequencies could be used that meet these same criteria. The 18 KHz tone was chosen so as to be well outside of the frequency range of the stethoscope audio data in order to reduce interference with the stethoscope data and preserve its fidelity. Obviously, other frequencies that meet these criteria could also be used.

As noted previously, a significant feature of the present invention is the ability to transmit audio data, particularly stethoscope data derived from an electronic stethoscope containing frequencies in the range of approximately 20 Hz-1600 Hz over a POTS line having a frequency response of approximately 300 Hz-3400 Hz. The stethoscope audio data can not be directly transmitted over a POTS line without experiencing significant degradation of the data. The invention overcomes this problem by modulating a carrier signal having a frequency that can be transmitted over a POTS line using the stethoscope audio data as the modulation signal.

The POTS passband is not wide compared to the desired transmission band, and therefore, the carrier frequency is inevitably close in frequency to the transmission band. In addition, a POTS line does not possess a flat frequency response resulting in the reception of different frequencies at different amplitude levels. Thus, some simple and conventional modulation transmission techniques are difficult to adapt.

The inventive modulation approach has many advantages when compared to other possible modulation schemes. The inventive modulation technique uses single-sideband transmission with a pilot carrier at one half the modulation frequency. In one embodiment, the modulation frequency is 2108 Hz, and a pilot tone at one half the carrier frequency (1054 Hz) is transmitted with the modulation signal. The upper sideband from 2120 Hz to 3400 Hz is transmitted. With this particular arrangement, the pilot tone and upper sideband are easily separated at the receiver which uses the pilot tone to generate the local oscillator. A phase-locked-loop (PLL) is used to synchronize the local oscillator to the modulated signal for direct demodulation of the modulated carrier to the baseband signal. One advantage of the particular modulation/demodulation approach is that synchronous demodulation is utilized and thus the modulator and demodulator at the respective local site and remote site are synchronized so that stethoscope data can be transmitted over a POTS line in real time with minimal distortion.

The motivation to send a separate pilot tone arises from the significant energy content below 30 Hz present in the stethoscope audio signals. When modulated, these low frequency signals appear as spectral components closely clustered about the modulation frequency. A PLL trying to lock on to the carrier in the presence of the closely clustered sidebands experiences significant phase jitter which appears in the demodulated signal. When a pilot tone at one half the carrier frequency is used, the pilot tone is spectrally located away from the significant audio energy of the stethoscope signal. A low pass filter can then be used to separate the pilot tone from the signals clustered about the modulation frequency. The PLL may then be used to generate a synchronized local oscillator signal at twice the pilot tone frequency. The chosen carrier frequency of 2108 Hz allows straightforward separation of the pilot tone from the upper sideband. The carrier frequency of 2108 Hz provides a signal bandwidth of approximately 1300 Hz for transmission of stethoscope audio signals over a POTS line. Since the stethoscope audio signals range in frequency from approximately 20 Hz to approximately 1600 HZ, the 1300 Hz bandwidth allows transmission of substantially all of the signals of interest over a POTS line.

Transmission of the upper sideband (USB) is advantageous when compared to transmission of the lower sideband (LSB) because the frequency of the pilot tone falls within the lower sideband. If the lower sideband were transmitted, baseband signals within the signal pass band of the stethoscope might interfere with the PLL of the receiver, resulting in distortion of the output signal. Respiratory and heart signals from the stethoscope with a baseband frequency around approximately 1054 Hz appear, after modulation at 2108 Hz, within the lower sideband approximately around the pilot tone frequency. The signals may result in jitter in the PLL output.

Choosing a pilot tone frequency at one half the carrier frequency is advantageous due to the ease of generating the two frequencies with a binary frequency relationship. This is also advantageous because it is then straightforward to reconstruct the local oscillator frequency at twice the pilot tone frequency. However, other relationships between the carrier frequency and the pilot tone frequency are certainly within the scope of the invention. Other choices of frequency relationships between the carrier frequency and the pilot tone frequency that might place the pilot tone frequency within the upper sideband may require more complex circuitry. Some empirical testing using the lower sideband resulted in distortion of the output signal when the input signal contained frequency components near the pilot tone frequency. Using the upper sideband avoids this distortion.

One of ordinary skill in the art will recognize that a system that transmits the lower sideband of the modulated carrier signal and a pilot tone above the carrier frequency (where the upper sideband would be) can also be implemented. Additionally, with the aforementioned limitations, one can implement a double sideband transmission system where no pilot tone is generated, and the carrier frequency is also the modulation frequency. One of ordinary skill in the art will appreciate that, on the receiving side, a corresponding reception system for the lower sideband transmission system or the double sideband transmission system can be implemented.

Reference is now made to FIG. 8A, which figure is a schematic block diagram of modulator 182 illustrated in FIG. 7. The combination of modulator 182 and demodulator 206 allows the present invention to carry out the modulation method just described.

Modulator 182 receives the stethoscope audio data on line 184 and provides a modulated output signal on line 186. The modulator includes a clock generator 250 which generates both the 2108 Hz carrier frequency on line 252 and the 1054 Hz pilot tone frequency on line 254. The 1054 Hz square wave pilot tone frequency passes through low pass filter 256. Low pass filter 256 is a four pole Butterworth filter having a corner frequency at approximately 900 Hz. The 2108 Hz square wave carrier frequency on line 252 is routed through a low pass filter 258. Low pass filter 258 is a four pole Butterworth filter having a corner frequency at approximately 1800 Hz. Low pass filters 256 and 258 are used to reduce the amplitude of any odd harmonic overtones present in the square wave signals.

The stethoscope audio data on line 184 is routed to a pre-emphasis filter 260. The pre-emphasis filter 260 creates a zero at 20 Hz and a pole at 500 Hz so that the signal content through the frequency band is increasingly boosted. A corresponding de-emphasis filter 262 in demodulator 206 attenuates these frequencies in an inverse manner in order to restore the original signal balance. The pre-emphasis filter approximately matches the input signal energy to the communications channel capacity in order to improve the signal-to-noise ratio of the transmitted signal. The filtered carrier frequency on line 264 and the filtered stethoscope audio signal on line 266 are provided to modulator 268 in which the 2108 Hz carrier frequency is modulated by the stethoscope audio signal. The output of modulator 268 on line 270 is provided to a single sideband high pass filter 272. Filter 272 is a 16 pole elliptical filter having a corner frequency at approximately 2010 Hz. The output of filter 272 on line 274 is provided to a summing circuit 276. The filtered pilot tone signal on line 278 is also provided to summing circuit 276. Summing circuit 276 sums the output of the single sideband filter 272 and the output of low pass filter 256 and provides this signal on line 280 to variable gain amplifier 282. The output of amplifier 282 on line 284 is provided to a low pass filter 286. Low pass filter 286 is a four pole Butterworth filter having a corner frequency at approximately 3600 Hz and is used to remove unwanted frequency content that might produce alias frequencies within the POTS network. The low pass filtered output signal on line 186 is then provided to multiplexer 188 illustrated in FIG. 7.

Reference is now made to FIG. 8B, which figure is a schematic block diagram of demodulator 206 illustrated in FIG. 5.

Demodulator 206 receives the modulated stethoscope audio signal from the telephone system on line 204 and provides the modulated stethoscope audio signal to circuits 302 and 304. Circuit 304 is used to provide the pilot tone on line 306. Circuit 304 is used to provide the modulated stethoscope audio signal on line 308.

In circuit 302, the filtered modulated stethoscope audio signal on line 204 is provided to a variable gain amplifier 310. The output of amplifier 310 is provided on line 312 to low pass filter 314. Low pass filter 314 is a four pole Butterworth filter having a corner frequency at approximately 900 Hz. Setting the corner frequency at approximately 900 Hz results in a filter slope of approximately 24 dB per octave at 1054 Hz in order to provide additional rejection of the 2108 Hz carrier frequency compared to the 1054 Hz pilot tone. The pilot tone frequency provided on line 316 is then provided to phase shifter 318. Phase shifter 318 shifts the signal through nearly 180 degrees of phase shift in order to maximize the demodulated signal amplitude. Typically this phase shift is used for initial calibration and does not require readjustment. The phase shifted output from phase shifter 318 is provided on line 320 to frequency doubler 322. Frequency doubler 322 doubles the 1054 Hz pilot tone frequency to provide a synthetic carrier at 2108 Hz on line 324. The synthetic carrier on line 324 is then provided to phase-locked-loop 326. The output of the phase-locked-loop is then provided on line 306 to demodulator 328.

In circuit 304, the filtered modulated stethoscope audio signal on line 204 is provided to a variable gain amplifier 330. The output of variable amplifier 330 on line 332 is provided to high pass filter 334. High pass filter 334 is a four pole Butterworth filter having a corner frequency at approximately 1800 Hz. The output of filter 334 is provided on line 336 to a notch filter 338. Notch filter 338 has a 3 dB bandwidth of 20 Hz and provides approximately 35 dB of rejection at 1054 Hz. The combination of filters 334 and 338 remove the 1054 Hz pilot tone from the modulated audio signal.

The output of circuit 302 on line 306 and the output of 304 on line 308 are provided to demodulator 328. The demodulated output signal on line 340 is provided to de-emphasis filter 262. De-emphasis filter 262 attenuates high frequency noise which has been added to the signal by the POTS line. Filter 262 has a pole at 20 Hz and a zero at 500 Hz. When combined with the pre-emphasis filter 260 in modulator 182, the combined response of the pre-emphasis filter 260 and the de-emphasis filter 262 provides a flat frequency response from approximately 20 Hz to approximately 1600 HZ within approximately 1 dB.

The output of de-emphasis filter 262 on line 342 is provided to a notch filter 344. A notch filter 344, having a frequency notch at 1054 Hz, further reduces any remaining pilot tone signal. The output of notch filter 344 on line 346 is provided to low pass filter 348. Low pass filter 348 is an eight pole elliptical filter having approximately 0.3 dB ripple. Low pass filter 348 has a corner frequency of approximately 1400 Hz. The restored baseband stethoscope audio signal from filter 348 is then provided on line 207.

Reference in now made to FIG. 9 which figure is a schematic diagram of an illustrative circuit embodiment of the block diagram of FIG. 6. The illustrated circuit can be powered by a single 9 volt battery. The integrated circuits used in the circuit of FIG. 9 are listed below:

Integrated Circuit List (FIG. 9)

| IC# | Part# | Manufacturer | Description |
|---|---|---|---|
| U1 | LM567 | National Semiconductor | tone detector |
| U2 | 4053 | Harris Semiconductor | multiplexer |
| U3 | ZSR330C | Toko America | voltage regulator |
| U4 | LT1013 | Linear Technology | op amp |
| U5 | TLE2425 | Texas Instruments | virtual ground generator |

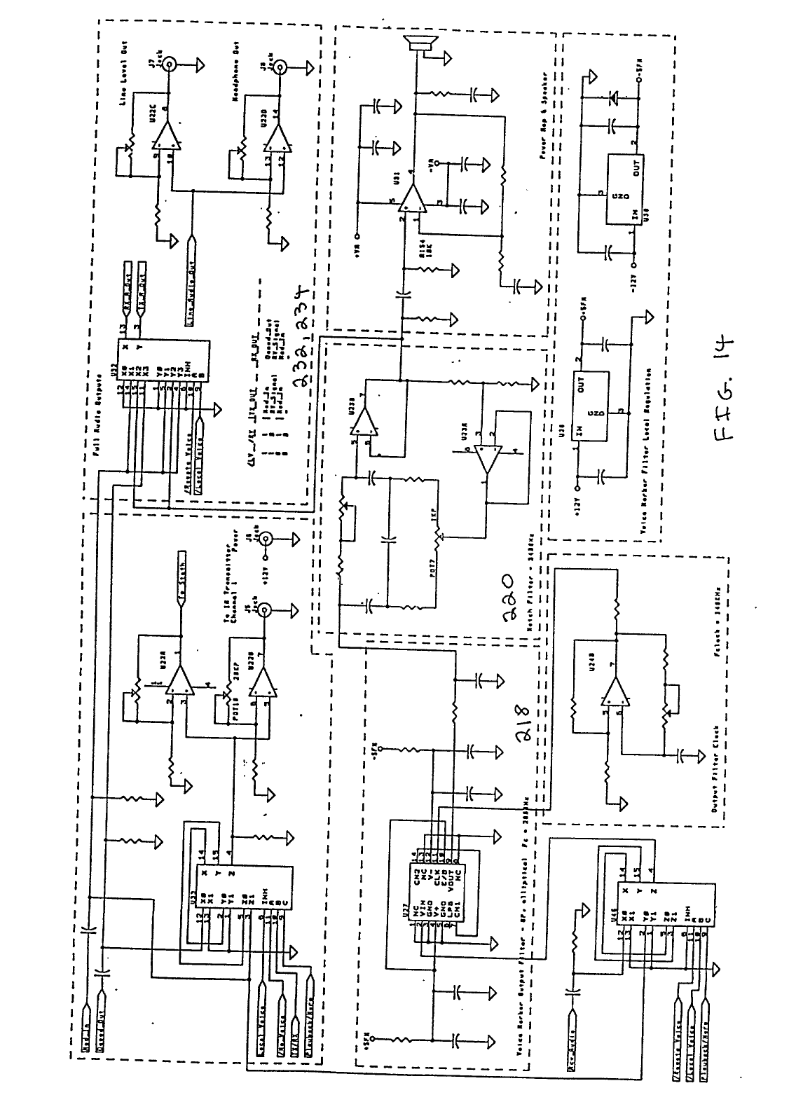

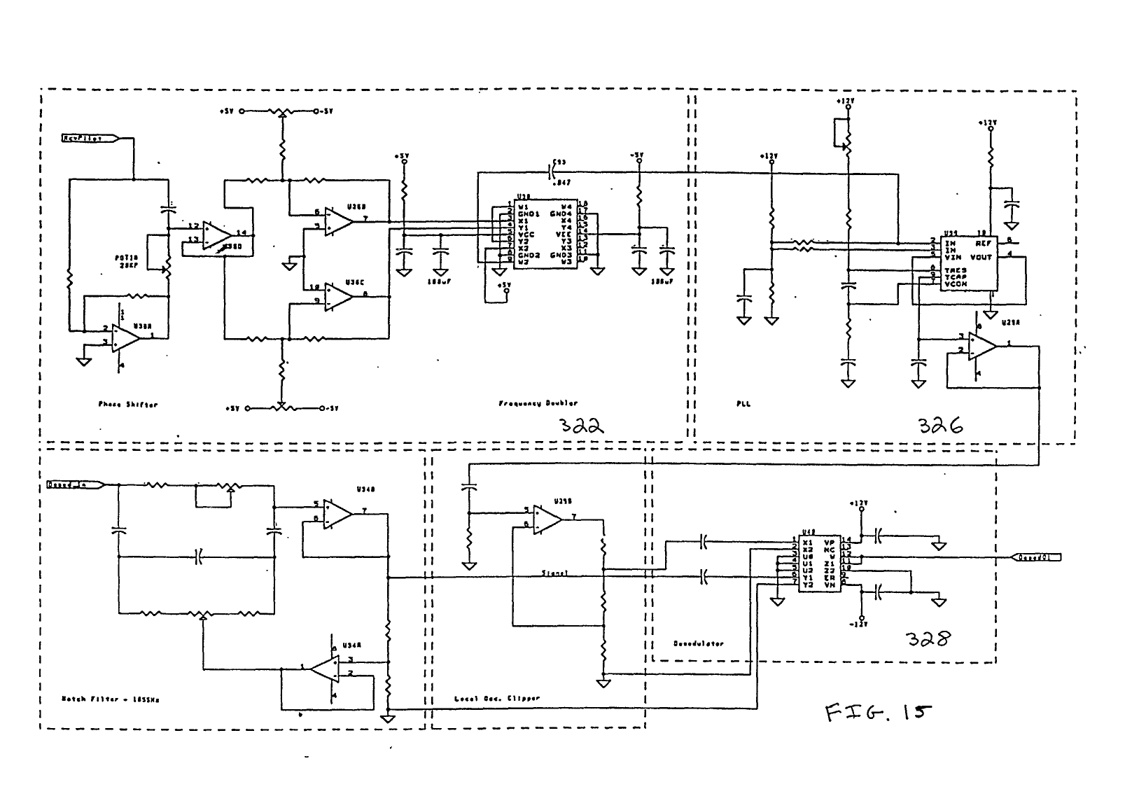

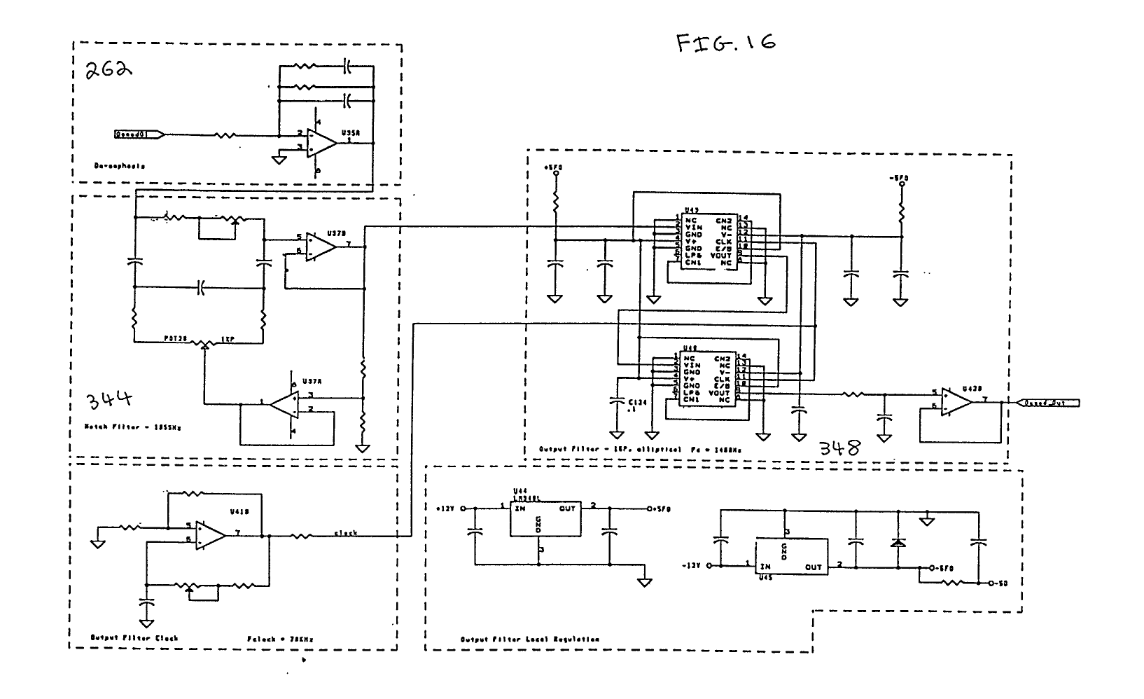

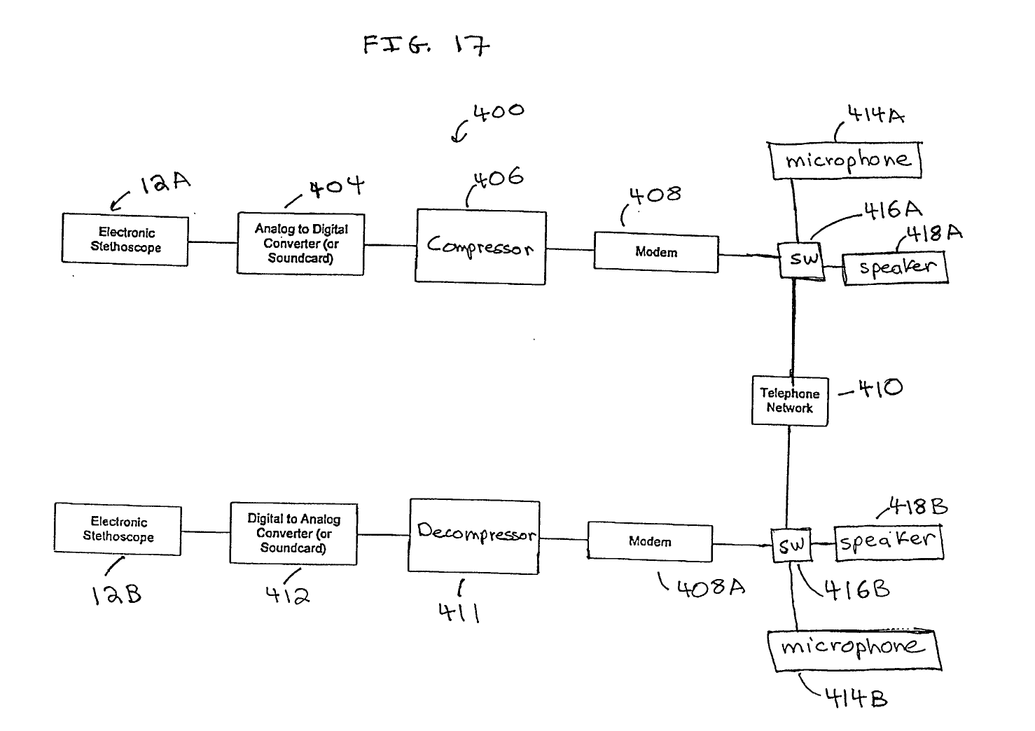

Reference is now made to FIGS. 10, 11, 12, 13, 14, 15, and 16 which figures are a schematic diagram of an illustrative circuit embodiment of the block diagrams of FIGS. 7 and 8. The integrated circuits used in the circuit of FIGS. 10, 11, 12, 13, 14, 15, and 16 are listed below:

Integrated Circuit List (FIGS. 10, 11, 12, 13, 14, 15, and 16)

| IC# | Part# | Manufacturer | |
|---|---|---|---|
| Description | | | |
| U1, U6, U9, U11, | LF412 | National Semiconductor | op amp |
| U13, U17, U23, U24 | | | |
| U29, U34, U35, U37, | | | |
| U41, U42 | | | |
| U2, U7, U33, U46 | 4053 | Harris Semiconductor | |
| multiplexer | | | |
| U3, U25 | LM567 | National Semiconductor | tone |
| detector | | | |
| U4 | 4011 | Harris Semiconductor | nand gate |
| U5 | ZSR330C | Toko America | voltage |
| regulator | | | |
| U8, U19, U20, U21, | LE347 | National Semiconductor | op amp |
| U22, U36 | | | |
| U10 | 4060 | National Semiconductor | counter/ |
| divider | | | |
| U12, U40 | AD734 | Analog Devices | multiplier |
| U14, U16 | LTC1068 | Linear Technology | filter |
| U15, U28, U44 | LM340L | National Semiconductor | voltage |
| regulator | | | |
| U18, U30, U45 | LM320L | National Semiconductor | voltage |
| regulator | | | |
| U27, U43, U46 | LTC- | Linear Technology | filter |
| | 1164-6 | | |
| U31 | LM1875 | National Semiconductor | power |
| amp | | | |
| U32 | 4052 | Harris Semiconductor | |
| multiplexer | | | |
| U38 | MLT04 | Analog Devices | multiplier |
| U39 | LM565C | National Semiconductor | PLL |

The present invention has been illustrated with circuitry for transmitting stethoscope audio signals having a frequency range of 20 Hz to 1300 Hz. However, the frequency response of the system can be increased to provide a transmission bandwidth of 20 Hz to 1600 Hz by changing the parameters of the frequencies used in modulator 182 and demodulator 206. For example, if the carrier frequency was set to approximately 1800 Hz, the pilot tone frequency was set to approximately 900 Hz, the corner frequency of filter 256 was changed to approximately 750 Hz, the corner frequency of filter 258 was changed to approximately 1550 Hz, the corner frequency of single-sideband filter 272 was changed to approximately 1800 Hz and the corner frequency of low pass filter 286 was changed to approximately 3500 Hz, then modulator 182 would be capable of transmitting signals having a bandwidth of 20 Hz to approximately 1600 Hz.

In the same manner, in demodulator 206, changing the corner frequency of low pass filter 314 to approximately 750 Hz, the corner frequency of high pass filter 334 to approximately 1550 Hz, the notch frequency of notch filter 338 to approximately 900 Hz, the notch frequency of notch filter 344 to approximately 900 Hz, and the corner frequency of low pass filter 348 to approximately 1600 Hz, allows demodulator 206 to receive and demodulate signals having a bandwidth of approximately 20 Hz to approximately 1600 Hz.

Thus far, the present invention has been particularly explained and illustrated using an analog implementation. The analog technique for real-time transmission over a single telephone line of analog electrical signals representative of audio signals derived from an electronic stethoscope has several advantages. It has compatibility with any standard voice quality telephone line; it can be built from low-cost analog components; and it can facilitate simple switching back and forth between stethoscope signal transmission and voice transmission.

An alternative approach to transmission of stethoscope and voice signals over a conventional POTS line is to use a digital methodology. This technique takes an analog representation of the stethoscope signal and digitizes the signal through an analog to digital (A/D) converter. Then the output signal of the A/D is compressed with high speed digital hardware. The compressed digital signal is transmitted over the telephone line using a digital modem. Voice transmissions are also compressed and sent over the telephone line in a similar fashion.

A benefit of digital transmission is that a transmitted signal of known quality will result in a received signal of equal quality from a telephone channel of certain minimum transmission characteristics. If the telephone channel will support modem communication at a certain data rate, then this channel can be relied upon to provide a precise reproduction of the transmitted signal at the receiving end of the transmission. Transmitted signal quality will depend upon the compression characteristics and not directly on channel characteristics. The compression algorithm provides a representation of the signal that is compatible with the channel data rate.

Another benefit of digital transmission is that a circuitry required to implement the system is mostly digital and therefore inherently stable. The analog approach, in contrast, requires the use of some particularly stable and precise components to achieve the same level of stability.

Stethoscope audio signals can contain information over a bandwidth of 20 Hz to 1600 Hz. The Nyquist sampling theorem indicates that in order to reconstruct an analog signal from a set of digital samples of that signal the sampling frequency must be at least twice the frequency of the highest frequency component of the sampled signal. The use of a ratio of at least 2.2 of the sample frequency to highest signal frequency makes the construction of band-limiting filters simpler. These filters are used to eliminate the aliases that might be present in the reconstructed signal.

Experience has shown that a sample resolution of 13 bits is appropriate for the representation of stethoscope audio data. There is no noticeable improvement in audio fidelity above this resolution; however, there is a perceptible degradation in audio fidelity if a lower resolution is used. 13 bit resolution is also accepted as the standard for high quality speech signal processing.

The inherent data rate of the full stethoscope audio band is therefore:

1600 cycles/second*2.2 samples/cycle*13 bits/sample=45,760 bits/second

The highest speed modem currently available for use on a conventional telephone line provides a transmission rate of 33,600 bits/second. This upper limit on data rate transmission is a result of telephone line characteristics and could only be surpassed with the replacement of significant portions of the telephone network itself. Many telephone lines can only be counted on to provide a low error communication channel at 14,400 bits/second, and some can only support a transmission at 9,600 bits/second. A low error channel is one where the error rate does not produce a perceptible change in the transmitted signal. Because standard modem protocols utilize a start and stop bit, the available data transmission bit rate is reduced by 20% from the aforementioned numbers.

In order to achieve real-time transmission of stethoscope audio, compression ratios of 2:1, 4:1, and 6:1 are required at modem rates of 28.8, 14.4 and 9.6 kbits/second, respectively. Since the stethoscope communication system is designed to be used on the most basic POTS line, it is desirable for the data rate to support 9.6 kbits/second for the worst quality lines. The compression algorithms described below will provide multiple levels of compression and will operate at the lowest compression rate compatible with the transmission rate supported by a particular call connection over a telephone line.

Logarithmic compression is an amplitude compression technique that provides greater resolution to a signal at lower amplitude than at higher amplitude, corresponding to the sensitivity of human hearing. American and European standards define μ- Law and A-Law transformations which are commonly applied to signals transmitted over standard telephone networks. Common commercial compression/decompression integrated circuits (CODECS) provide digitization and compression consistent with these standards. These devices digitize a signal with a 13 bit dynamic range and provide an 8 bit digital result. It has been determined empirically that stethoscope audio signals suffer no appreciable loss of fidelity due to this amplitude compression. Using this technique, however, does not achieve the necessary level of compression required for modem communication.

Since sampled stethoscope signals contain a high degree of correlation from one sample to the next, it may be possible to achieve significant compression of sampled data by providing, as a data set, the difference between samples instead of the sample itself. This technique is call Differential Pulse Code Modulation (DPCM). It has been determined empirically that when an original stethoscope data set is represented with 8 bits of resolution the difference set derived from the original set will contain no values larger than would be represented by 5 bits. Using a combination of logarithmic compression and DPCM, the 13 bit data set can be compressed to a 5 bit set. This yields a compression ratio of 2.6:1 which will support digital transmission over a 28.8 kbits/second modem link.

DPCM achieves compression by making the assumption that a good estimate for the value of the signal is the value it had at the previous sample and that the error from this estimate is statistically smaller than the distance of the signal from zero. By using more of the signal than just the previous sample, a better prediction of the next value can be achieved. If both the compressing and the decompressing ends of the communication know what prediction would result from a previous sequence of samples, then only the difference from this prediction needs to be transmitted. Commonly, the predicted value is a linear combination of a previous number of samples. This technique is known as Linear Predictive Coding. To produce a transmitted value, each of n previous samples is multiplied by a corresponding prediction coefficient, and the products are summed to produce the next predicted value. The difference between this predicted value and the actual value is transmitted. Better compression is achieved by using more samples to create a predicted value, but both signal delay and computation requirements increase with more samples.

In one common form of compression used in telephony, the prediction coefficients and quantization interval are continually modified on the basis of signal history. This technique is known as Adaptive Differential Pulse Code Modulation (ADPCM). CCITT and ANSI define algorithms which support voice band to 3400 Hz and produce 2 and 4 bit 8 ksample/second data streams achieving effective compression ratios of 6.5:1 and 3.25:1. A compression algorithm based upon this approach and modified for the stethoscope bandwidth and signal characteristic achieves the necessary compression ratio for transmission over a 9600 kbits/second modem channel. A corresponding decompression algorithm is used at the receiver. ADPCM compression is one method by which a 6.5:1 compression ratio can be achieved. 6.5:1 compression is needed to allow the maximum data rate for the lowest common denominator telephone lines which typically can sustain a data rate of 9600 kbits/second. However any compression algorithm that can provide at least a 6:1 compression ratio can be used in the present invention.

Reference is now made to FIG. 17, which figure illustrates one embodiment of a digital implementation of the present invention. An electronic stethoscope 12A provides a signal to an analog-to-digital converter 404. Analog-to-digital converter 404 converts the analog signal from the analog data signal from electronic stethoscope 12A into a digital signal with, for example, 13 bit resolution. Analog-to-digital converter 404 provides the 13 bit digital signal to compressor 406. Compressor 406 uses a compression algorithm to compress the digital data from analog-to-digital converter 404 by a compression ratio of at least 6:1. The digital data from compressor 406 is then provided to modem 408 which transmits the digital data over telephone network 410. Analog-to-digital converter 404 and compressor 406 may be incorporated into a computer. Compressor 406 may be implemented in either hardware or software. In one embodiment, compressor 406 uses an ADPCM compression algorithm to produce 2 bit 8 ksample/second data streams achieving an effective compression ratio of 6.5:1. This compression ratio allows the stethoscope signals to be transmitted over a POTS line having a minimum transmission bandwidth of 9600 kbits/second.

From telephone network 410, the signal is received by modem 408A which sends the received signal to decompressor 411. Decompressor 411 executes a decompression algorithm that decompresses the digital data to restore the same data stream provided to compressor 406. In one embodiment decompressor 411 uses an ADPCM decompression algorithm. From decompressor 411, the decompressed data is provided to digital-to-analog converter 412 that converts the digital data to an analog data signal that is then provided to electronic stethoscope 12B. Digital-to-analog converter 412 and decompressor 411 can be part of a computer and decompressor 411 can be implemented in hardware or software as described in connection with compressor 406.

A switch 416A which may be, for example, a “push-to-talk” switch, is provided that switches between the digital data provided by modem 408 or 408A or the voice signal from microphone 414A or 414B (to be routed to speakers 418B or 418A, respectively). In one embodiment the normal mode of operation, switch 416A selects the digital data signal from modem 408 and controls switch 416B to send the digital data to modem 408A. In the voice override mode of operation, switch 416A selects the voice signal from microphone 414A and controls switch 416B to disconnect modem 408A and send the voice signal to speaker 418B. In the same manner, switch 416B can switch between the digital data provided by modem 408 or 408A or the voice signal from microphone 414A or 414B (to be routed to speakers 418B or 418A, respectively).

The switching system can, in one embodiment, be programmed to emulate the switching that is implemented in the embodiment of the invention illustrated in FIGS. 1-16. Other switching protocols are also possible.

One skilled in the art will appreciate that the voice signals could also be digitized and routed through compressor 406 and decompressor 411. ADPCM compression and decompression algorithms can be used with the voice signal.

The hardware for a transmitter for digitization of stethoscope signals is built around a dedicated microcontroller such as the Microchip PIC17C42 and a conventional modem such as the Hayes Optima 288. Alternatively, the system can be built using an IBM-compatible PC with an Intel 486 or Pentium processor. Either system would implement an ADPCM compression algorithm, provide a connection to the telephone line, contain a user interface to facilitate operation, and provide an interface (using a sound card) directly to the electronic stethoscope or through the aforementioned IR communications link to the stethoscope via a mobile unit.

Another use of the present invention is to allow an EMT or other medical practitioner when in the field to transmit patient heart and/or lung sounds to another more skilled medical practitioner at a remote location (e.g. the hospital to which the patient will be transported) in real time for diagnosis and triage instruction using the electronic stethoscope in combination with the present invention and a cellular telephone coupled to a single conventional telephone line.

Another use of the present invention is to allow heart and/or lung sounds to be recorded locally or remotely for subsequent diagnosis and/or for comparison to previous or subsequent examinations of the same patient. The recorded data can be annotated with either or both of the local or remote users' comments during the examination for later reference. This can be accomplished using the electronic stethoscope in combination with the present invention to provide a local recording. A remote recording can be accomplished using the electronic stethoscope in combination with the present invention and a single conventional telephone line.

There are many benefits to the present invention. One benefit of the present invention is that it gives physicians and other medical practitioners instant access to other remotely located (and presumably more skilled) physicians or diagnosticians anywhere in the world via conventional telephone lines for remote real time diagnosis.

Another benefit of the present invention is that real-time diagnosis of an intermittent or critical problem can be accomplished without moving the patient. Using current technology, a patient would have to be sent to the specialist which could be impractical, time-consuming, or even life-threatening in order for the specialist to examine the patient. In critical care situations this would not necessarily be possible especially if the patient were critically injured in the field. In the case of some intermittent problem, the abnormal sound might not be present when the patient subsequently went to the specialist to be examined at a later date or time. Using the present invention, the patient can potentially obtain an instant diagnosis remotely from any doctor anywhere in the world.

Another benefit of the present invention is its ability to reduce the cost of providing medical care. This can be accomplished in many ways. A general practitioner does not necessarily have to send a patient to a hospital or other remote facility for more expensive testing when a heart or lung abnormality is suspected; rather the general practitioner can allow a more skilled specialist to listen to the patient's heart and/or lungs remotely to determine if there really is a reason for the patient to be examined and tested by the specialist. In the case of bed-ridden or nursing home patients for example, this can obviate an expensive ambulance ride to a hospital where the patient will typically be administered to in an emergency room setting which is a much more costly method of accomplishing this kind of diagnosis. Another potential cost savings involves patients with certain conditions such as chronic asthma, chronic obstructive pulmonary disease (COPD), emphysema, congestive heart failure, or other conditions which potentially could require a patient to stay several days or even weeks in the hospital at a very high cost simply so that a physician could listen to the patient several times per day with his or her stethoscope to monitor the condition of the patient. Using the present technology, many of these patients could stay at home and could be examined remotely by the physician several times a day at the physician's discretion.

Given the current climate in the healthcare industry which has become very cost conscious, the present invention provides many advantages over the current state-of-the-art technology. In addition, with health maintenance organizations (HMO's) and other healthcare providers and insurers trying to reduce costs by using lesser skilled doctors or nurses for screening patients to avoid sending them to specialists, the present technology can enable the resources of the healthcare provider to be substantially increased. Less skilled medical practitioners can provide routine examinations that can be remotely confirmed by greater skilled specialists who do not have to be physically present with the patient for each examination.

Another benefit of the present invention is its ability to create a permanent record of the auscultatory data and findings for comparison to a previous and/or future examination. Current acoustic stethoscope technology does not allow the physician to record any auscultatory data. Therefore, the physician must rely on memory, which is not particularly accurate or reliable, to evaluate the potential progress or decline of a patient's condition. The present invention enables the physician to make direct comparisons between and among two or more examinations of the patient's heart and/or lung sounds to assess the progress of the patient's condition.

Having thus described at least one illustrative embodiment of the invention, various alterations, modifications, and improvements will readily occur to those skilled in the art. Such alterations, modifications, and improvements are intended to be within the spirit and scope of the invention. Accordingly, the foregoing description is by way of example only and is not intended as limiting. The invention is limited only as defined in the following claims and the equivalents thereto.

What is claimed is:

1. A communication and remote diagnosis system, comprising:
a first electronic stethoscope that provides a data signal having a first bandwidth;
a first base unit that transmits the data signal through a telephone line having a second bandwidth;
a communication link that couples the data signal provided by the electronic stethoscope to the first base unit;
wherein the first base unit includes a generator circuit that generates a carrier signal having a frequency within the second bandwidth and a modulator, coupled to the generator circuit, that modulates the carrier signal with the signal having the first bandwidth to provide a modulated carrier signal;
a first interface circuit, coupled to the first base unit, that injects the modulated carrier signal into the telephone line;
a second interface circuit, coupled to the telephone line, that receives the modulated carrier signals and provides a received modulated carrier signal; and
a second base unit, coupled to the second interface circuit, including a demodulator that demodulates the received modulated carrier signal to provide the signal having the first bandwidth.

2. The communication and remote diagnosis system of claim 1, wherein the generator circuit generates a pilot tone having a frequency lower than the frequency of the carrier signal and the first base unit further comprises:
a filter, coupled to the modulator, that filters the modulated carrier signal to remove lower sideband signals to provide a filtered modulated carrier signal; and
a summing circuit, coupled to the modulator and to the generator circuit, that combines the filtered modulated carrier signal with the pilot tone signal to provide an output signal;
wherein the first interface circuit injects the output signal into the telephone line.

3. The communication and remote diagnosis system of claim 2, wherein the frequency of the pilot tone is approximately one half the frequency of the carrier signal.

4. The communication and remote diagnosis system of claim 1, wherein the generator circuit generates a pilot tone having a frequency greater than the frequency of the carrier signal and the first base unit further comprises:
a filter, coupled to the modulator, that filters the modulated carrier signal to remove upper sideband signals to provide a filtered modulated carrier signal; and
a summing circuit, coupled to the modulator and to the generator circuit, that combines the filtered modulated carrier signal with the pilot tone signal to provide an output signal;
wherein the first interface circuit injects the output signal into the telephone line.

5. The communication and remote diagnosis system of claim 1, wherein the second base unit further comprises:
a first circuit that processes the received modulated carrier signal to provide a pilot tone signal having a first frequency;
a second circuit that processes the first frequency to provide a carrier signal; and
a third circuit that filters the received modulated carrier signal to remove a pilot tone frequency to provide a received filtered modulated carrier signal;
wherein the demodulator is coupled to the second and third circuits and demodulates the received filtered modulated carrier signal to provide the signal having the first bandwidth.

6. The communication and remote diagnosis system of claim 5, wherein the second circuit multiplies the first frequency to provide the carrier signal.

7. The communication and remote diagnosis system of claim 6, wherein the second circuit doubles the first frequency.

8. The communication and remote diagnosis system of claim 5, wherein the second circuit divides the first frequency to provide the carrier signal.

9. The communication and remote diagnosis system of claim 1, further comprising means for selecting one of the data signal or a voice signal to be transmitted through the telephone line.

10. The communication and remote diagnosis system of claim 1, further comprising a second electronic stethoscope that receives the signal having the first bandwidth and a second communication link that couples the signal having the first bandwidth from the second base unit to the second electronic stethoscope.

11. The communication and remote diagnosis system of claim 1, wherein at least one of the first and second communication links is an infrared communications link.

12. The communication and remote diagnosis system of claim 1, wherein at least one of the first and second communication links is a radio frequency communications link.

13. The communication and remote diagnosis system of claim 1, wherein the first bandwidth is in the range of approximately 20 Hz to approximately 1600 Hz.

14. The communication and remote diagnosis system of claim 13, wherein the second bandwidth is in the range of approximately 300 Hz to approximately 3400 Hz.

15. The communication and remote diagnosis system of claim 1, wherein at least one of the first and second communication links includes a transceiver.

16. The apparatus of claim 15, wherein the transceiver is incorporated into at least one of the first and second electronic stethoscopes.

17. The communication and remote diagnosis system of claim 1, wherein the frequency of the pilot tone is within the second bandwidth.

18. A system for transmitting a signal having a first bandwidth through a telephone line having a second bandwidth, wherein the first bandwidth includes frequencies outside the second bandwidth, the system comprising:
a first electronic stethoscope that provides a signal having the first bandwidth;
an analog-to-digital converter, coupled to the first electronic stethoscope, that converts the signal to a first digital signal;
a compressor, coupled to the analog-to-digital converter, that compresses the first digital signal by a ratio so as to allow transmission of the first digital signal through the telephone line to provide a second digital signal; and
a first modem, coupled to the compressor, that injects the second digital signal into the telephone line.

19. The apparatus of claim 18, wherein the compressor compresses the digital signal by a ratio of at least 6 to 1.

20. The apparatus of claim 19, wherein the compressor uses an ADPCM compression algorithm.

21. The apparatus of claim 20, wherein the compressor compresses the first digital signal by a ratio of 6.5 to 1.

22. The system of claim 18, further comprising:
a second modem, coupled to the telephone line, that receives the second digital signal from the telephone line;
a decompressor, coupled to the second modem, that decompresses the second digital signal by a ratio that restores the first digital signal;
a digital-to-analog converter, coupled to the decompressor, that converts the first digital signal to an analog signal having the first bandwidth; and
a second electronic stethoscope that receives the analog signal having the first bandwidth.

23. The apparatus of claim 22, wherein the decompressor decompresses the second digital signal by a ratio of at least 1 to 6.

24. The apparatus of claim 23, wherein the decompressor uses an ADPCM decompression algorithm.

25. The apparatus of claim 24, wherein the decompressor decompresses the second digital signal by a ratio of 1 to 6.5.

26. A stethoscope to stethoscope communication system, the system comprising:
a first electronic stethoscope that provides an electronic signal representative of biological activity;
a transmitter, coupled to the first electronic stethoscope, that transmits the electronic signal;
at least one receiver that receives the transmitted electronic signal; and at least one additional electronic stethoscope coupled to the at least one receiver.

27. The stethoscope to stethoscope communication system of claim 26, wherein the transmitter and the at least one receiver use an infrared communication link.

28. The stethoscope to stethoscope communication system of claim 26, wherein the transmitter and the at least one receiver use a radio frequency communication link.

29. The communication and remote diagnosis system of claim 1, further comprising an analog-to-digital converter constructed and arranged to digitize the signal having the first bandwidth.

30. The apparatus of claim 22, further comprising means for transmitting a voice signal through the telephone line.

31. The apparatus of claim 30, wherein the means for transmitting includes a compressor and a decompressor.

32. The apparatus of claim 31, wherein the compressor and decompressor use ADPCM algorithms.
